(19) United States
(12) Patent Application Publication
(10) Pub. No.: US 2006/0111313 A1
(43) Pub. Date: May 25, 2006

(54) Nell-1 enhanced bone mineralization

(76) Inventor: Kang Ting, Beverly Hills, CA (US)

Correspondence Address: SQUIRE, SANDERS & DEMPSEY L.L.P., 1 MARITIME PLAZA, SUITE 300, SAN FRANCISCO, CA, 94111, US

(21) Appl. No.: 10/527,786

(22) PCT Filed: Sep. 15, 2003
(86) PCT No.: PCT/US03/29281

§ 371 Date: Sep. 28, 2005

Related U.S. Application Data
(60) Provisional application No. 60/410,846, filed on Sep. 13, 2002.

(51) Int. Cl. A61K 48/00

(52) U.S. Cl. 514/44

(57) ABSTRACT

This invention pertains to the discovery that the human NELL-1 gene induces or upregulates bone mineralization. The HELL-1 gene or gene product thus provides a convenient target for screening for modulators of bone mineralization. In addition, HELL-1 can be used to facilitate repair of bone fractures and/or to generally increase bone density.

### CROSS-REFERENCE TO RELATED APPLICATIONS

This application claims priority to and benefit of U.S. Ser. No. 60/410,846, filed on Sep. 13, 2002, which is incorporated herein by reference in its entirety for all purposes.

### STATEMENT AS TO RIGHTS TO INVENTIONS MADE UNDER FEDERALLY SPONSORED RESEARCH AND DEVELOPMENT

This work was supported by NIH/NIDR grant number DE94001 and CRC/NIH grant number RR00865. The Government of the United States of America may have certain rights in this invention.

### FIELD OF THE INVENTION

This invention relates to the discovery that upregulation of NELL-1 enhances bone calcification. NELL-1 thus provides a good target to screen for modulators of bone calcification. In addition, NELL-1 proteins can be used in a manner analogous to bone morphogenic proteins to facilitate bone repair.

### BACKGROUND OF THE INVENTION

Craniosynostosis (CS), the premature closure of cranial sutures, affects 1 in 3,000 infants and therefore is one of the most common human congenital craniofacial deformities (1). Premature suture closure, which results in cranial dysmorphism, can be either familial or sporadic in origin (1). Neither gender nor ethnicity can be used to predict which infants will be affected. Although genetic linkage analyses of CS-related syndromes have provided a wealth of new information about the molecular control of suture formation, the biology of local suture closure, especially in nonsyndromic, nonfamilial CS, is still largely unknown.

Presently, more than 85 human mutations, which produce various familial CS syndromes, have been localized to the FGF receptor genes FGFR1, FGFR2, and FGFR3. All are “gain-of-function” mutations that result in increased receptor activity (1). No human CS syndromes have been linked to the FGP ligands; however, several animal models of CS have been associated with FGF overexpression (2, 3). The only described MSX2 mutation associated with CS (4) also results in increased MSX2 activity (5-7). While these candidate genes are known to play important roles in osteoblast proliferation and differentiation, they also have more generalized roles during embryogenesis. Thus, it is not surprising that transgenic mouse models with mutations in these genes often manifest extracranial abnormalities not observed in the majority of patients with CS (1, 2, 8).

Premature suture closure in human CS can be divided into two possibly distinct processes: calvarial overgrowth and bony fusion. While calvarial overgrowth may be essential to bringing the two opposing osteogenic fronts into proximity in order to induce bony fusion, it does not necessarily follow that calvarial overgrowth or overlap alone will result in bony fusion. Thus, the study of premature suture closure mechanisms must include study of both abnormal suture overgrowth/overlap and bony fusion (6).

Recently, FGF2 and FGFR1 have been implicated in premature cranial suture fusion via CBFA1-mediated pathways (8). Missense mutation of CBFA1 is linked to cleidocranial dysplasia, manifested as delayed suture closure (9). Therefore, examination of Cbfa1 (Runx2), a downstream target of Fgfr1 that is essential for bone formation, may be key to understanding the signaling cascade in CS. In addition, Msx2, a member of the highly conserved Msx homeobox gene family with pleiotropic effects in development, has been implicated in an animal model of CS (5, 6). Specifically, increased osteogenic cell proliferation has been proposed as a mechanism for premature suture closure in Msx2-overexpressing transgenic mice, which exhibit suture overgrowth/overlap without suture fusion.

### SUMMARY OF THE INVENTION

To elucidate the molecular pathway for suture closure, we previously used differential display to identify genes that were specifically upregulated within abnormally fused sutures in patients with nonfamilial, nonsyndromic CS. We isolated and characterized NELL-1, which is a Nel-like, type 1 molecule (a protein strongly expressed in neural tissue, encoding an EGF-like domain) (1012). Nell-1 is a secreted protein. Structurally, Nell-1 encodes a secretory signal peptide sequence, an NH2-terminal thrombospondin-1-like module, five von Willebrand factor-like repeats with six cysteine residues, and six EGFlike domains. Nell-1 is also highly conserved across species. For example, 93% amino acid sequence homology exists between rat Nell-1 and human NELL-1.

Nell-1 encodes a polypeptide with a molecular weight of 90 kDa. When overexpressed in COS cells, the glycosylated form is N-linked to a 50-kDa carbohydrate moiety in eukaryotic cells to generate the 140-kDa form found in the cytoplasm. This 140-kDa protein is further processed to a 130-kDa protein. The Nell-1 protein is secreted as a trimeric form with a high molecular weight (approximately 400 kDa) (13, 14).

Initial studies have suggested that NELL-1 is preferentially expressed in the craniofacial region of calvarial tissues (12-14). Premature suture closure in CS patients is remarkable for the degree of NELL-1 overexpression by osteoblast-like cells in osteogenic areas (12). Although Nell-1 overexpression and premature suture closure may be coincidental findings, our data suggest that Nell-1 may be a local regulatory factor in cranial suture closure. In this study, we further verified that Nell-1 has a role in CS. We created a transgenic mouse model exhibiting generalized Nell-1 overexpression. Nell-1 transgenic animals share many of the same features as humans with CS. They demonstrate calvarial overgrowth/overlap and premature suture closure. Infection of osteoblasts with Nell-1 adenoviral constructs showed that Nell-1 promotes and accelerates differentiation in osteoblast lineage cells. In addition, Nell-1 downregulation inhibited osteoblast differentiation. Nell-1, therefore, represents a candidate gene for producing cranial suture closure and provides new insights in the study of CS and craniofacial development.

In one embodiment, this invention provides methods of modulating calvarial osteoblast differentiation and mineralization. The methods involve altering expression or activity of Nell-1, where increased expression or activity of Nell-1 increases osteoblast differentiation or mineralization and decreased expression or activity of Nell-decreases osteoblast differentiation or mineralization. Nell-1 expression or activity is can be inhibited by any convenient method (e.g. an anti-Nell-1 antisense molecule, a Nell-1 specific ribozyme, a Nell-1 specific catalytic DNA, a Nell-1 specific RNAi, anti-Nell-1 intrabodies, and gene therapy approaches that knock out Nell-1 in particular target cells and/or tissues). Similarly, Nell-1 expression or activity can be increased by any convenient method (e.g. by transfecting a cell with an exogenous nucleic acid expressing Nell-1, transfecting a cell with a Nell-1 protein, etc.). The mammal can be a mammal (human or non-human mammal) experiencing abnormal cranial suture development (e.g. Craniosynostosis (CS)).

This invention also provides a method of facilitating latent TGF-β1 activation in a mammal. The method can involve administering exogenous Nell-1 to the mammal, or increasing expression activity of endogenous Nell-1 in said mammal.

Also provides is a method of activating or sequestering a member of the TGF-β superfamily in a mammal. The method involves administering exogenous Nell-1 to teh mammal, or increasing expression activity of endogenous Nell-1 in the mammal.

In still another embodiment, this invention provides methods of screening for an agent that modulates osteoblast differentiation. The methods involve contacting a test cell containing a NELL-1 gene with a test agent; and detecting a change in the expression level of a NELL-1 gene or the activity of Nell-1 in the test cell as compared to the expression of the NELL-1 gene or the activity of Nell-1 in a control cell where a difference in the expression level of NELL-1 or the activity of Nell-1 in the test cell and the control cell indicates that said agent modulates bone mineralization. In certain embodiments, the control is a negative control cell contacted with the test agent at a lower concentration (e.g. half concentration, absense of test agent, etc.) than the test cell. In certain embodiments, the control is a positive control cell contacted with the test agent at a higher concentration than the test cell. In various embodijments, the expression level of nell-1 is detected by measuring the level of NELL-1 mRNA in said cell and/or the level of NELL-1 is detected by determining the expression level of a NELL-1 protein in the biological cell, e.g. as described herein.

In still another embodiment, this invention provides methods of altering Nell-1 expression in a mammalian cell. The methods involve altering the expression or activity of Msx2 and/or Cbfa1. In certain embodiments, Cbfa1 expression or activity is upregulated to upregulate Nell-1 expression or activity. In certain embodiments, Msx2 expression or activity is upregulated to downregulate Nell-1 expression or activity.

Similarly, methods are provided for screening for an agent that modulates Nell-1 expression or activity, said method comprising where the methods involve contacting a test cell containing a Cbfa1 and/or an Msx2 gene with a test agent; and detecting a change in the expression level of an Cbfa1 and/or an Msx2gene or the activity of Cbfa1 and/or an Msx2 in said test cell as compared to the expression of the Cbfa1 and/or an Msx2 gene or the activity of Cbfa1 and/or an Msx2 in a control cell where a difference in the expression level of Cbfa1 and/or an Msx2or the activity of Cbfa1 and/or an Msx2 in the test cell and the control cell indicates that the agent modulates Nell-1 expression or activity.

Also provided is a pharmaceutical formulation, comprising: one or more active agents selected from the group consisting of a nucleic acid encoding a Nell-1 protein, a Nell-1 protein, and an agent that alters expression or activity of a Nell-1 protein; and a pharmaceutically acceptable excipient.

In other embodiments, this invention pertains to the discovery that the polypeptide encoded by the human NELL-1 gene induces bone mineralization and is therefore osteogenic. The NELL-1 gene and gene product(s) (e.g. mRNA, cDNA, protein, etc.) provide good targets for screening for modulators of NELL-1 expression and/or activity and therefore for modulators of bone mineralization. In addition, NELL-1 can be used in a manner analogous to the use of bone morphogenic proteins (BMPs) to speed fracture repair and as a component of bone graft materials.

As indicated, in one preferred embodiment, this invention provides methods of screening for an agent that alters bone mineralization. The methods involve contacting a cell containing a NELL-1 gene with a test agent; and detecting a change in the expression level of the NELL-1 gene as compared to the expression of the NELL-1 gene in a cell that is not contacted with the test agent where a difference in the expression level (e.g. as represented by genomic DNA copy number, mRNA level, protein level, protein activity, etc.), of NELL-1 in the contacted cell and the cell that is not contacted indicates that said agent modulates bone mineralization. The methods may further involve test agents that alter expression of the NELL-1 nucleic acid or the NELL-1 protein in a database of modulators of NELL-1 activity or in a database of modulators of bone mineralization. In certain embodiments, the expression level of NELL-1 is detected by measuring the level of NELL-1 mRNA in the cell (e.g. by hybridizing said mRNA to a probe that specifically hybridizes to a NELL-1 nucleic acid). Preferred hybridization methods include, but are not limited to a Northern blot, a Southern blot using DNA derived from the NELL-1 RNA, an array hybridization, an affinity chromatography, and an in situ hybridization. The methods of this invention are amenable to array-based approaches. Thus, in some embodiments, the probe is a member of a plurality of probes that forms an array of probes. The level of NELL-1 expression can also be determined using a nucleic acid amplification reaction (e.g. PCR).

In other embodiments of the screening systems of this invention, NELL-1 expression is detected by determining the expression level of a NELL-1 protein (e.g. via of capillary electrophoresis, a Western blot, mass spectroscopy, ELISA, immunochromatography, immunohistochemistry, etc.) in the biological sample. The cell can be cultured ex vivo or can be in vivo and/or in situ. In certain embodiments, the test agent is not an antibody and/or not a protein and/or not a nucleic acid. Preferred test agents are small organic molecules.

This invention also provides methods of prescreening for a potential modulator of NELL-1 expression and/or activity. The methods involve contacting a NELL-1 nucleic acid or a NELL-1 protein with a test agent; and detecting specific binding of said test agent to the NELL-1 protein or nucleic acid. The method can further involve recording test agents that specifically bind to the NELL-1 nucleic acid or to said NELL-1 protein in a database of candidate modulators of NELL-1 activity and/or in a database of candidate modulators of bone mineralization. The test agent can be contacted directly to the NELL-1 nucleic acid and/or protein, or to a cell and/or tissue and/or organism (e.g., mammal) containing the nucleic acid and/or protein. Where a cell is contacted, the cell can be in a primary or passaged culture. In certain embodiments, the test agent is not an antibody and/or not a protein and/or not a nucleic acid. Preferred test agents are small organic molecules. Where the assay measures the ability of the test agent to bind to a nucleic acid, preferred assays utilize a Northern blot, a Southern blot using DNA, an array hybridization, an affinity chromatography, or an in situ hybridization. Where the assay measures the ability of the test agent to bind to a NELL-1 protein, preferred assays utilize capillary electrophoresis, a Western blot, mass spectroscopy, ELISA, immunochromatography, or immunohistochemistry).

In another embodiment, this invention provides methods of increasing bone mineralization. Preferred methods involve increasing the concentration of a NELL-1 gene product in an osteogenic cell (e.g. an osteoblast, a mesenchymal cell, a fibroblast cell, a fetal embryonic cell, a stem cell, a bone marrow cell, a dura cell. a chrondrocyte, a chondroblast, etc.) or in the milieu within which the cell is found. In one preferred embodiment, the concentration of NELL-1 gene product is increased by upregulating expression of a NELL-1 gene. This can be accomplished by any of a wide variety of methods including, but not limited to upregulating expression of an endogenous NELL-1 gene (e.g. by modifying the endogenous regulatory region e.g. the promoter), or transfecting the cell with a vector that expresses a NELL-1 protein. Certain preferred vectors constitutively expresses a NELL-1 protein, while other preferred vectors are inducible. In still another embodiment, the NELL-1 gene product concentration is increased by the bone with a NELL-1 polypeptide.

This invention also provides methods of facilitating the repair of bone fractures. These methods involve increasing concentration of a NELL-1 gene product at or near the fracture site. In preferred embodiments, the NELL-1 gene product is increased in an osteogenic or bone precursor cell present at or near the fracture site. The methods can involve introducing an osteogenic cell or bone precursor cell that overexpresses NELL-1 into the fracture site. In another embodiment, this invention can involve increasing the concentration of a NELL-1 gene product in the osteogenic cell or said bone precursor cell in situ. The NELL-1 gene product up-regulation can be achieved as described herein. In another embodiment, the cell and/or bone fracture site is contacted with a NELL-1 polypeptide.

In another approach to fracture repair, the fracture site is contacted with a NELL-1 protein. The protein can be produced by a cell (e.g. introduced by introduction of a cell overexpressing NELL-1 protein) and/or by administration of the protein alone or in combination with a pharmacological excipient, and/or by administration of a “naked DNA” vector capable of expressing NELL-1. The NELL-1 protein can be a component of a bone repair/bone graft material and/or part of a prosthetic device. One preferred graft material includes collagen and/or bone fragments in addition to the NELL-1 protein and/or cells expressing a NELL-1 protein.

In still yet another embodiment this invention provides a bone graft material capable of enhancing the formation of osseous tissue in the animal in which it is implanted. Preferred bone graft materials consist essentially of a biocompatible matrix and a NELL-1 protein. A preferred graft material is resorbable/biodegradeable. Again, the matrix can be impregnated with a NELL-1 protein and/or a cell expressing a NELL-1 protein. A preferred bone graft material comprises a collagen conjugate containing: (e.g., about 65 to about 95 weight percent) reconstituted collagen having dispersed substantially uniformly therein; and (e.g., about 35 to about 5 weight percent) a NELL-1 protein and/or a cell expressing a NELL-1 protein.

### DEFINITIONS

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The terms “NELL-1 cDNA” and “NELL-1” genomic DNA refer to the cDNA and genomic DNA as disclosed by Watanabe et al. (1996) Genomics 38 (3): 273-276; Ting et al. (1999) J Bone Mineral Res, 14: 80-89; and GenBank Accession Number U57523).

A NELL-1 protein is a protein expressed by the NELL-1 gene or cDNA. The NELL-1 protein can include NELL-1 protein fragments that retain the ability to induce bone mineralization.

The term “antibody”, as used herein, includes various forms of modified or altered antibodies, such as an intact immunoglobulin, an Fv fragment containing only the light and heavy chain variable regions, an Fv fragment linked by a disulfide bond (Brinkmann et al. (1993) Proc. Natl. Acad. Sci. USA, 90: 547-551), an Fab or (Fab)′2 fragment containing the variable regions and parts of the constant regions, a single-chain antibody and the like (Bird et al. (1988) Science 242: 424426; Huston et al. (1988) Proc. Nat. Acad. Sci. USA 85: 5879-5883). The antibody may be of animal (especially mouse or rat) or human origin or may be chimeric (Morrison et al. (1984) Proc Nat. Acad. Sci. USA 81: 6851-6855) or humanized (Jones et al. (1986) Nature 321: 522-525, and published UK patent application #8707252).

The terms “binding partner”, or “capture agent”, or a member of a “binding pair” refers to molecules that specifically bind other molecules to form a binding complex such as antibody-antigen, lectin-carbohydrate, nucleic acid-nucleic acid, biotin-avidin, etc.

The term “specifically binds”, as used herein, when referring to a biomolecule (e.g., protein, nucleic acid, antibody, etc.), refers to a binding reaction which is determinative of the presence biomolecule in heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

The term osteoporosis refers to a heterogeneous group of disorders characterized by decreased bone mass and fractures. Clinically, osteoporosis is segregated into type I and type II. Type I osteoporosis occurs predominantly in middle aged women and is associated with estrogen loss at the menopause, while osteoporosis type II is associated with advancing age.

Osteogenesis imperfecta (OI) refers to a group of inherited connective tissue diseases characterized by bone and soft connective tissue fragility (Byers & Steiner (1992) Annu. Rev. Med. 43: 269-289; Prockop (1990) J. Biol. Chem. 265: 15349-15352). Males and females are affected equally, and the overall incidence is currently estimated to be 1 in 5,000-14,000 live births. Hearing loss, dentinogenesis imperfecta, respiratory insufficiency, severe scoliosis and emphysema are just some of the conditions that are associated with one or more types of 01. While accurate estimates of the health care costs are not available, the morbidity and mortality associated with OI certainly result from the extreme propensity to fracture (OI types I-IV) and the deformation of abnormal bone following fracture repair (OI types II-IV).

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10): 1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 141 9), phosphorothioate (Mag et al. (1991) Nucleic Acids Res. 19:1437; and U.S. Pat. No. 5,644,048), phosphorodithioate (Briu et al. (1989) J. Am. Chem. Soc. 111 :2321, O-methylphophoroamidite linkages (see Eckstein, Oligonucleotides and Analogues: A Practical Approach, Oxford University Press), and peptide nucleic acid backbones and linkages (see Egholm (1992) J. Ant. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences. Stringent hybridization and stringent hybridization wash conditions in the context of nucleic acid hybridization are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, chapt 2, Overview of principles of hybridization and the strategy of nucleic acid probe assays, Elsevier, N.Y. (Tijssen). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook (1989) Molecular Cloning: A Laboratory Manual (2nd ed.) Vol. 1-3, Cold Spring Harbor Laboratory, Cold Spring Harbor Press, NY, and detailed discussion, below), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook supra.) for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 40° C. for 15 minutes.

“Osteogenic cells” are cells capable of mineralizing. Osteogenic cells include osteoblasts, osteoblast like cells, mesenchymal cells, fibroblast cells, fetal embryonic cells, stem cells, bone marrow cells, dura cells. chrondrocyes, and chondroblastic cells.

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term database refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

### BRIEF DESCRIPTION OF THE DRAWINGS

FIG. 1A illustrates over-expression of NELL-1 in E-14 rat calvarial primary cell cultures using adenoviruses with β-galactosidase as control. FIG. 1B shows a plot of mineralization as a function of time post treatment with NELL-1 and β-glactosidase respectively. Experiments were performed in triplicate. Student's T test was performed. Mineralization with NELL-1 was statistically higher than mineralization with β-Galactosidase control, *P<0.001.

FIG. 2 illustrates Nell-1 transgenic mice compared with nontransgenic littermates. (FIG. 2A) Transgene copy number. The founders (FA and FB) and their progeny (TF₂A1, TF₂A2, and TF₂B1) have copy numbers between 50 and 100. TF₂A1 and TF₂A2 are from the founder A line. TF₂B1, TF₂B2, and TF₂B3 are from the founder B line. (FIG. 2B) RT-PCR analyses of Nell-1 RNA expression in both founders. C, control Nell-1 plasmid; M, muscle; H, heart; B, bone; K, kidney; L, liver. (FIG. 2C) Whole body (without head) RNA of newborn progeny. TF2A1 and TF2A2 express different levels of Nell-1. TF2B1 expresses Nell-1 weakly, while TF2B2 and TF2B3 have no Nell-1 expression. (FIG. 2D) Left panels, immunolocalization of Nell-1 protein in newborn NF2 epithelium, muscle, and calvarial bone. There is no detectable Nell-1 expression (brown staining indicates the presence of Nell-1) except some staining in the calvarial bone. Right panels, immunolocalization of Nell-1 protein in TF2A2 epithelium, muscle, and calvarial bone. Abundant Nell-1 expression is present throughout all soft tissue layers as well as in bone. Bar represents 50 μm.

FIG. 3 Phenotypic evaluation of Nell-1 transgenic mice. (a and b) Left panels show a newborn Nell-1 phenotype-positive (TF2A1) mouse. Note the protrusion in the frontoparietal area (arrows). Right panels show an NF2 littermate. (c) Left panel, TF2A2 mouse with the scalp removed. The sagittal (yellow arrow) and PF (black arrow) sutures are closed. Right panel, skull of the NF2 littermate with patent sagittal (yellow arrows) and PF (black arrow) sutures and normal vasculature underneath the patent sutures. (d) An infant with craniotelencephalic dysplasia, a severe form of CS. (e) Brain MRI of TF₂A1 mouse (left) and NF2 littermate (right). Note the complete absence of ventricles, suggesting elevated intracranial pressure in the TF₂A1 mouse (arrows, left) relative to its NF2 littermate (arrows, right). (f) MCT-reconstructed threedimensional skull views of the newborn Nell-1 phenotypepositive TF₂A1 (left) and NF2 (right) littermates. Arrows indicate sagittal and PF suture sites. In TF₂A1 mice, the sagittal and PF sutures are largely closed and replaced with an abnormal ridge. In the NF2 littermate, both sagittal and PF sutures are patent. Complete opacity corresponds to greater than 50 mg/cc mineralization. The vertical rods in the background are phantom reference rods corresponding to mineralization densities (from left to right) of 50, 100, 150, and 200 mg/cc. (g) Serial axial MCT sections of the TF2A1 (left) and NF2 littermates (right) shown in f. Yellow arrows indicate the distortion of the cranium. Green arrows indicate increased mineralization of the calvarium in the TF2A mouse (arrow, right).

FIG. 4 Histologic and immunohistologic evaluation of Nell-1 transgenic mice. (Panel a) Hematoxylin and eosin staining of the sagittal suture of a Nell-1 phenotype-positive TF2A1 mouse. There is closure of the suture, shown by the overlap of calvarial edges (black arrows) and closing osteogenic fronts (red arrows). Lower left panel shows von Kossa staining. Note the close proximity of mineralized calvarial edges. (Panel b) Hematoxylin and eosin staining of the sagittal suture from an NF2 littermate. Note the large distance separating the two calvarial edges (black arrows) at the patent suture site, as well as the advancing osteogenic fronts (red arrows). Lower left panel shows von Kossa staining. Black color indicates mineralization. Panel c) Immunolocalization of alkaline phosphatase (ALP) in a TF2A1 mouse. Brown staining indicates the presence of alkaline phosphatase (arrows). Lower panel represents the immunolocalization of osteopontin at lower magnification. (Panel d) Upper panel shows immunolocalization of alkaline phosphatase in newborn NF2 cranial suture. Lower panel represents the immunolocalization of osteopontin (OP) at a lower magnification. Bar represents 50 μm. (Panel e) BrdU staining of a TF2 sagittal suture. The nuclei of proliferating cells are stained brown (black arrows). Proliferating cells are significantly decreased relative to those shown for NF2 in d. (Panel f) BrdU staining of a newborn NF2 mouse sagittal suture. Numerous brownstained cells are proliferating along the calvarial edges (black arrows) of the patent suture, as well as along the advancing osteogenic fronts (red arrows). H&E, hematoxylin and eosin. (Panel g) Number of proliferating cells per field.

FIG. 5 Nell-1 transgenic TF₂B1 mouse compared with a nontransgenic littermate. (a) Left, newborn TF2B1 mouse with the scalp removed. Note the abnormal bulging of the occipital area and the relatively narrow width of the cranium. Right, an NF2 littermate. In the TF2B1 transgenic animal, the sagittal suture and several other sutures are closed. (b) Hematoxylin and eosin staining of TF2B1 sagittal suture. Premature closure of the suture is manifest in the severe overlap of calvarial edges (red arrows). The underlying brain tissue has been removed for RNA analysis. (c) Three-dimensional MCT reconstruction of a TF2B1 mouse (left) and its NF2 littermate (right). Note the area of premature midline suture closure in the TF2B1 mouse (arrow, left).

FIG. 6 illustrates the effects of Nell-1 overexpression on mineralization and bone marker expression. (FIG. 6A) FRCC culture infected with 20 pfu/cell AdNell-1, stained with von Kossa stain. Control cell cultures were infected with Ad β-Gal. Experiments were performed in triplicate. Mineralized nodules are stained black. (FIG. 6B) Quantitation and statistical analysis of mineralized areas. AdNell-1-infected cultures demonstrated significantly greater mineralization than did Ad β-Gal controls. (FIG. 6C) AdNell-1-infected MC3T3 cells grown without ascorbic acid. Typical micronodule appearance is shown. Right panel represents alkaline phosphatase staining of a micronodule. FIGS. 6D-F) Microarrays of AdNell-infected MC3T3 cells on postinfection days 6, 9, and 12, respectively. Gene expression intensities have been normalized using standardized housekeeping genes (HKGs). Hybridization intensities of AdNell-1-infected cells are represented on the y axis. Hybridization intensities of Ad β-Gal-infected cells are represented on the x axis. HKGs r2 represents the correlation of housekeeping genes (filled squares) between the two samples. ECMs r2 represents the correlation of candidate gene expression (open squares) between the two samples. A photograph of the microarray reading is attached in the upper left corner of each diagram. A twofold or greater upregulation is represented in red, while a twofold or greater downregulation is represented in green (g) Table summarizing genes with a difference in expression that is twofold higher or lower after AdNell-1 infection. The ratio is calculated as Nell-1/β-Gal. Col, collagen.

FIG. 7 illustrates the effect of Nell-1 downregulation on alkaline phosphatase expression and bone marker expression. (FIG. 7A) Western blot analysis of Nell-1 protein expression in rat FRCCs infected with 20 pfu/cell AdAntiNell-1 or Ad β-Gal control. Downregulation of approximately 60% is observed. (FIG. 7B) Alkaline phosphatase staining (in red) of FRCCs. AdAntiNell-1-infected cells have significantly less staining than do control and AdNell-1-infected cells. (FIG. 7C) Northern analyses of FRCCs on days 3, 6, 9, and 12 after infection. AdAntiNell-1-infected cells have significantly less osteocalcin and osteopontin expression. (FIG. 7D) Expression of osteocalcin (OC) and osteopontin (OP) measured by PhosphorImager and normalized by GAPDH.

FIG. 8 illustrates a hypothetical model of Nell-1 function in premature suture closure. Dashed line represents potential modulation.

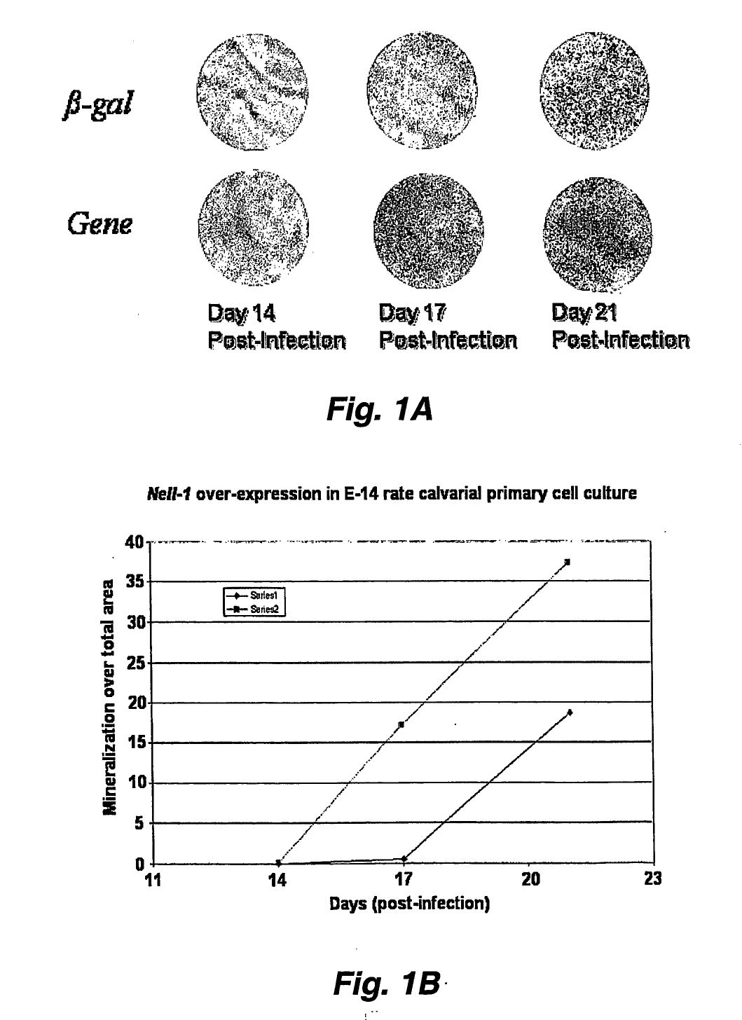

### DETAILED DESCRIPTION

This invention provides pertains to the discovery that the NELL-1 gene product enhances tissue (e.g. bone) mineralization. Without being bound to a particular theory, it is believed that the NELL-1 protein may execute its function by interacting with members of the TGFβ superfamily.

Having recognized that NELL-1 mediates tissue mineralization as described herein, NELL-1 nucleic acids and/or NELL-1 proteins provide convenient targets for screening for modulators of bone mineralization. Thus agents that inhibit NELL-1 expression and/or protein activity and/or protein-protein interactions will decrease bone mineralization. Conversely agents that upregulate NELL-1 expression and/or protein activity and/or protein-protein interactions are expected to increase bone mineralization. Such NELL-1 “agonists” are expected to be useful in a wide variety of contexts including, but not limited treatment of osteoporosis, bone fracture healing, treatment of osteogenesis imperfecta, bone reconstruction, and the like.

Thus, in one embodiment, this invention provides methods of identifying agents that modulate (e.g. up-regulate or down-regulate) NELL-1 expression. Such methods involve contacting a NELL-1 nucleic acid, and/or a cell containing a NELl-1 nucleic acid, and/or a tissue or organism comprising cells containing a NELL-1 nucleic acid and detecting changes in the level of NELL-1 transcript (e.g. mRNA) and/or NELL-1 protein. In one embodiment, candidate test agents for such an assay are identified in a binding assay “pre-screen”. Such a binding assay involves pre-screening test agent(s) for the ability to specifically bind to a NELL-1 nucleic acid and/or a NELL-1 protein. Agents identified by theses assays that upregulate NELL-1 expression are expected to be useful in the treatment of osteoporosis and bone fractures.

In addition, in a manner analogous to the use of bone morphogenic proteins (e.g. BMP-1 through BMP-24), the NELL-1 polypeptide(s) can be used to speed repair of bone fractures or to induce bone repair or replacement under circumstances where natural healing is limited or non-existent. In generally such methods involve increasing the amount of a NELL-1 gene product at or near the fracture site in a bone. The NELL-1 gene product concentration can be increased by one or more of a number of methods. In one approach, cells at or near the bone fracture site are induced to express elevated levels of NELL-1. This is accomplished, for example, by the use of modulators of NELL-1 expression, by altering the NELL-1 promoter, or by transfecting the cell with a construct that expresses NELL-1. This can be accomplished in vivo, or, in another embodiment, such cells can be modified to overexpress NELL-1 ex vivo and then introduced back into the subject organism (e.g. at or near a fracture site).

Cells expressing or overexpressing NELL-1 can be incorporated into bone graft materials and/or NELL-1 polypeptides can be incorporated into such bone graft materials. These graft materials can be used in the treatment of fractures or to facilitate the replacement/healing of prostheses or bone transplants.

I. Assays for Agents that Modulate NELL-1 expression.

As indicated above, in one aspect, this invention is premised on the discovery that NELL-1 mediates mineralization of bone and thus provides a good target for new agents that modulate bone mineralization. Thus, in one embodiment, this invention provides methods of screening for agents that modulate NELL-1 expression and hence bone mineralization. The methods involve detecting the expression level and/or activity level of a NELL-1 gene or gene product (e.g. NELL-1 protein) in the presence of the agent(s) in question. An elevated NELL-1 expression level or activity level in the presence of the agent as compared to a negative control where the test agent is absent or at reduced concentration indicates that the agent upregulates NELL-1 activity or expression. Conversely, decreased NELL-1 expression level or activity level in the presence of the agent as compared to a negative control where the test agent is absent or at reduced concentration indicates that the agent down-regulates NELL-1 activity or expression

Expression levels of a gene can be altered by changes in by changes in the transcription of the gene product (i.e. transcription of mRNA), and/or by changes in translation of the gene product (i.e. translation of the protein), and/or by post-translational modification(s) (e.g. protein folding, glycosylation, etc.). Thus preferred assays of this invention include assaying for level of transcribed mRNA (or other nucleic acids derived from the NELL-1 gene), level of translated protein, activity of translated protein, etc. Examples of such approaches are described below.

A) Nucleic-Acid Based Assays.

1) Target Molecules.

Changes in expression level can be detected by measuring changes in mRNA and/or a nucleic acid derived from the mRNA (e.g. reverse-transcribed cDNA, etc.). In order to measure the NELL-1 expression level it is desirable to provide a nucleic acid sample for such analysis. In preferred embodiments the nucleic acid is found in or derived from a biological sample. The term “biological sample”, as used herein, refers to a sample obtained from an organism or from components (e.g., cells) of an organism. The sample may be of any biological tissue or fluid. Biological samples may also include organs or sections of tissues such as frozen sections taken for histological purposes.

The nucleic acid (e.g., mRNA nucleic acid derived from mRNA) is, in certain preferred embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In a preferred embodiment, the “total” nucleic acid is isolated from a given sample using, for example, an acid guanidinium-phenol-chloroform extraction method and polyA+mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to assaying for expression level. Methods of amplifying nucleic acids are well known to those of skill in the art and include, but are not limited to polymerase chain reaction (PCR, see. e.g, Innis, et al., (1990) PCR Protocols. A guide to Methods and Application. Academic Press, Inc. San Diego,), ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA_{—}86: 1173), self-sustained sequence replication (Guatelli et al. (1990) Proc. Nat. Acad. Sci. USA 87: 1874), dot PCR, and linker adapter PCR, etc.).

In a particularly preferred embodiment, where it is desired to quantify the transcription level (and thereby expression) of NELL-1 in a sample, the nucleic acid sample is one in which the concentration of the NELL-1 mRNA transcript(s), or the concentration of the nucleic acids derived from the NELL-1 mRNA transcript(s), is proportional to the transcription level (and therefore expression level) of that gene. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes.

Where more precise quantification is required appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript or large differences of changes in nucleic acid concentration is desired, no elaborate control or calibration is required.

In the simplest embodiment, the NELL-1-containing nucleic acid sample is the total mRNA or a total cDNA isolated and/or otherwise derived from a biological sample. The nucleic acid may be isolated from the sample according to any of a number of methods well known to those of skill in the art as indicated above.

2) Hybridization-Based Assays.

Using the known sequence of NELL-1 (see, e.g., (SEQ ID NO: 1) detecting and/or quantifying the NELL-1 transcript(s) can be routinely accomplished using nucleic acid hybridization techniques (see, e.g., Sambrook et al. supra). For example, one method for evaluating the presence, absence, or quantity of NELL-1 reverse-transcribed cDNA involves a “Southern Blot”. In a Southern Blot, the DNA (e.g., reverse-transcribed NELL-1 mRNA), typically fragmented and separated on an electrophoretic gel, is hybridized to a probe specific for NELL-1. Comparison of the intensity of the hybridization signal from the NELL-1 probe with a “control” probe (e.g. a probe for a “housekeeping gene) provides an estimate of the relative expression level of the target nucleic acid.

Alternatively, the NELL-1 mRNA can be directly quantified in a Northern blot. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify and/or quantify the target NELL-1 mRNA. Appropriate controls (e.g. probes to housekeeping genes) provide a reference for evaluating relative expression level.

An alternative means for determining the NELL-1 expression level is in situ hybridization. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

In some applications it is necessary to block the hybridization capacity of repetitive sequences. Thus, in some embodiments, tRNA, human genomic DNA, or Cot-1 DNA is used to block non-specific hybridization.

3) Amplification-Based Assays.

In another embodiment, amplification-based assays can be used to measure NELL-1 expression (transcription) level. In such amplification-based assays, the target nucleic acid sequences (i.e., NELL-1 ) act as template(s) in amplification reaction(s) (e.g. Polymerase Chain Reaction (PCR) or reverse-transcription PCR (RT-PCR)). In a quantitative amplification, the amount of amplification product will be proportional to the amount of template (e.g., NELL-1 mRNA) in the original sample. Comparison to appropriate (e.g. healthy tissue or cells unexposed to the test agent) controls provides a measure of the NELL-1 transcript level.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). One approach, for example, involves simultaneously co-amplifying a known quantity of a control sequence using the same primers as those used to amplify the target. This provides an internal standard that may be used to calibrate the PCR reaction.

One preferred internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of labeled nucleic acid (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al. (1990) Academic Press, Inc. N.Y. The known nucleic acid sequence(s) for NELL-1 are sufficient to enable one of skill to routinely select primers to amplify any portion of the gene.

4) Hybridization Formats and Optimization of Hybridization

a) Array-Based Hybridization Formats.

In one embodiment, the methods of this invention can be utilized in array-based hybridization formats. Arrays are a multiplicity of different “probe” or “target” nucleic acids (or other compounds) attached to one or more surfaces (e.g., solid, membrane, or gel). In a preferred embodiment, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

This simple spotting, approach has been automated to produce high density spotted arrays (see, e.g., U.S. Pat. No: 5,807,522). This patent describes the use of an automated system that taps a microcapillary against a surface to deposit a small volume of a biological sample. The process is repeated to generate high density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high density arrays is also described in U.S. Pat. Nos. 5,744,305, 5,800,992 and 5,445,934.

b) Other Hybridization Formats.

As indicated above a variety of nucleic acid hybridization formats are known to those skilled in the art. For example, common formats include sandwich assays and competition or displacement assays. Such assay formats are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labelled probes or the like. Other labels include ligands that bind to labeled antibodies, fluorophores, chemi-luminescent agents, enzymes, and antibodies which can serve as specific binding pair members for a labeled ligand.

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art, are the nucleic acid sequence based amplification (NASBAO, Cangene, Mississauga, Ontario) and Q Beta Replicase systems.

c) Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

d) Labeling and Detection of Nucleic Acids.

The probes used herein for detection of NELL-1 expression levels can be full length or less than the full length of the NELL-1 mRNA. Shorter probes are empirically tested for specificity. Preferred probes are sufficiently long so as to specifically hybridize with the NELL-1 target nucleic acid(s) under stringent conditions. The preferred size range is from about 20 bases to the length of the NELL-1 mRNA, , more preferably from about 30 bases to the length of the NELL-1 mRNA, and most preferably from about 40 bases to the length of the NELL-1 mRNA.

The probes are typically labeled, with a detectable label. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a singie label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another.

Suitable chromogens which can be employed include those molecules and compounds which absorb light in a distinctive range of wavelengths so that a color can be observed or, alternatively, which emit light when irradiated with radiation of a particular wave length or wave length range, e.g., fluorescers.

Desirably, fluorescent labels should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye can differ from the unbound dye. Therefore, when referring to the various wavelength ranges and characteristics of the dyes, it is intended to indicate the dyes as employed and not the dye which is unconjugated and characterized in an arbitrary solvent.

Fluorescers are generally preferred because by irradiating a fluorescer with light, one can obtain a plurality of emissions. Thus, a single label can provide for a plurality of measurable events.

Detectable signal can also be provided by chemiluminescent and bioluminescent sources. Chemiluminescent sources include a compound which becomes electronically excited by a chemical reaction and can then emit light which serves as the detectable signal or donates energy to a fluorescent acceptor. Alternatively, luciferins can be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

The label may be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are easily added during an in vitro transcription reaction. Thus, for example, fluorescein labeled UTP and CTP can be incorporated into the RNA produced in an in vitro transcription.

The labels can be attached directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe—CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

B) Polypeptide-Based Assays.

1) Assay Formats.

In addition to, or in alternative to, the detection of NELL-1 nucleic acid expression level(s), alterations in expression of NELL-1 can be detected and/or quantified by detecting and/or quantifying the amount and/or activity of translated NELL-1 polypeptide.

2) Detection of Expressed Protein

The polypeptide(s) encoded by the NELL-1 can be detected and quantified by any of a number of methods well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmuno assay (RIA), enzyme-linked immunosorbent assays (ELISAs), immunofluorescent assays, western blotting, and the like.

In one preferred embodiment, the NELL-1 polypeptide(s) are detected/quantified in an electrophoretic protein separation (e.g. a 1- or 2-dimensional electrophoresis). Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another preferred embodiment, Western blot (immunoblot) analysis is used to detect and quantify the presence of polypeptide(s) of this invention in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target polypeptide(s) and may be directly labeled or alternatively may be subsequently detected using labeled antibodies (e.g., labeled sheep anti-mouse antibodies) that specifically bind to the a domain of the antibody.

In preferred embodiments, the NELL-1 polypeptide(s) are detected using an immunoassay. As used herein, an immunoassay is an assay that utilizes an antibody to specifically bind to the analyte (e.g., the target polypeptide(s)). The immunoassay is thus characterized by detection of specific binding of a polypeptide of this invention to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37. Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (NELL-1 polypeptide). In preferred embodiments, the capture agent is an antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/polypeptide complex.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Preferred immunoassays for detecting the target polypeptide(s) are either competitive or noncompetitive. Noncompetitive immunoassays are assays in which the amount of captured analyte is directly measured. In one preferred “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte (NELL-1 polypeptide) present in the sample is measured indirectly by measuring the amount of an added (exogenous) analyte displaced (or competed away) from a capture agent (antibody) by the analyte present in the sample. In one competitive assay, a known amount of, in this case, labeled polypeptide is added to the sample and the sample is then contacted with a capture agent. The amount of labeled polypeptide bound to the antibody is inversely proportional to the concentration of target polypeptide present in the sample.

In one particularly preferred embodiment, the antibody is immobilized on a solid substrate. The amount of target polypeptide bound to the antibody may be determined either by measuring the amount of target polypeptide present in an polypeptide/antibody complex, or alternatively by measuring the amount of remaining uncomplexed polypeptide.

The immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies that bind NELL-1 polypeptide(s), either alone or in combination. In the case where the antibody that binds NELL-1 polypeptide is not labeled, a different detectable marker, for example, an enzyme-labeled antibody capable of binding to the monoclonal antibody which binds the NELL-1 polypeptide, may be employed. Any of the known modifications of EIA, for example, enzyme-linked immunoabsorbent assay (ELISA), may also be employed. As indicated above, also contemplated by the present invention are immunoblotting immunoassay techniques such as western blotting employing an enzymatic detection system.

The immunoassay methods of the present invention may also be other known immunoassay methods, for example, fluorescent immunoassays using antibody conjugates or antigen conjugates of fluorescent substances such as fluorescein or rhodamine, latex agglutination with antibody-coated or antigen-coated latex particles, haemagglutination with antibody-coated or antigen-coated red blood corpuscles, and immunoassays employing an avidin-biotin or strepavidin-biotin detection systems, and the like.

The particular parameters employed in the immunoassays of the present invention can vary widely depending on various factors such as the concentration of antigen in the sample, the nature of the sample, the type of immunoassay employed and the like. Optimal conditions can be readily established by those of ordinary skill in the art. In certain embodiments, the amount of antibody that binds NELL-polypeptides is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2M sodium chloride, preferably about that of 0.15M sodium chloride. Times will vary widely depending upon the nature of the assay, and generally range from about 0.1 minute to about 24 hours. A wide variety of buffers, for example PBS, may be employed, and other reagents such as salt to enhance ionic strength, proteins such as serum albumins, stabilizers, biocides and non-ionic detergents may also be included.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein, are commercially available or can be produced as described below.

3) Antibodies to NELL-1 Polypeptides.

Either polyclonal or monoclonal antibodies may be used in the immunoassays of the invention described herein. Polyclonal antibodies are preferably raised by multiple injections (e.g. subcutaneous or intramuscular injections) of substantially pure polypeptides or antigenic polypeptides into a suitable non-human mammal. The antigenicity of the target peptides can be determined by conventional techniques to determine the magnitude of the antibody response of an animal that has been immunized with the peptide. Generally, the peptides that are used to raise antibodies for use in the methods of this invention should generally be those which induce production of high titers of antibody with relatively high affinity for target polypeptides encoded by NELL-1.

If desired, the immunizing peptide may be coupled to a carrier protein by conjugation using techniques that are well-known in the art. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g. a mouse or a rabbit).

The antibodies are then obtained from blood samples taken from the mammal. The techniques used to develop polyclonal antibodies are known in the art (see, e.g., Methods of Enzymology, “Production of Antisera With Small Doses of Immunogen: Multiple Intradermal Injections”, Langone, et al. eds. (Acad. Press, 1981)). Polyclonal antibodies produced by the animals can be further purified, for example, by binding to and elution from a matrix to which the peptide to which the antibodies were raised is bound. Those of skill in the art will know of various techniques common in the immunology arts for purification and/or concentration of polyclonal antibodies, as well as monoclonal antibodies see, for example, Coligan, et al. (1991) Unit 9, Current Protocols in Immunology, Wiley Interscience).

Preferably, however, the antibodies produced will be monoclonal antibodies (“mAb's”). For preparation of monoclonal antibodies, immunization of a mouse or rat is preferred. The term “antibody” as used in this invention includes intact molecules as well as fragments thereof, such as, Fab and F(ab′)^{2′}, and/or single-chain antibodies (e.g. scFv) which are capable of binding an epitopic determinant. Also, in this context, the term “mab's of the invention” refers to monoclonal antibodies with specificity for a polypeptide encoded by a NELL-1 polypeptide.

The general method used for production of hybridomas secreting mAbs is well known (Kohler and Milstein (1975) Nature, 256:495). Briefly, as described by Kohler and Milstein the technique comprised isolating lymphocytes from regional draining lymph nodes of five separate cancer patients with either melanoma, teratocarcinoma or cancer of the cervix, glioma or lung, (where samples were obtained from surgical specimens), pooling the cells, and fusing the cells with SHFP-1. Hybridomas were screened for production of antibody which bound to cancer cell lines. Confirmation of specificity among mAb's can be accomplished using relatively routine screening techniques (such as the enzyme-linked immunosorbent assay, or “ELISA”) to determine the elementary reaction pattern of the mAb of interest.

Antibodies fragments, e.g. single chain antibodies (scFv or others), can also be produced/selected using phage display technology. The ability to express antibody fragments on the surface of viruses that infect bacteria (bacteriophage or phage) makes it possible to isolate a single binding antibody fragment, e.g., from a library of greater than 10¹⁰ nonbinding clones. To express antibody fragments on the surface of phage (phage display), an antibody fragment gene is inserted into the gene encoding a phage surface protein (e.g., pill) and the antibody fragment-pIII fusion protein is displayed on the phage surface (McCafferty et al. (1990) Nature, 348: 552-554; Hoogenboom et al. (1991) Nucleic Acids Res. 19: 4133-4137).

Since the antibody fragments on the surface of the phage are functional, phage bearing antigen binding antibody fragments can be separated from non-binding phage by antigen affinity chromatography (McCafferty et al. (1990) Nature, 348: 552-554). Depending on the affinity of the antibody fragment, enrichment factors of 20 fold-1,000,000 fold are obtained for a single round of affinity selection. By infecting bacteria with the eluted phage, however, more phage can be grown and subjected to another round of selection. In this way, an enrichment of 1000 fold in one round can become 1,000,000 fold in two rounds of selection (McCafferty et al. (1990) Nature, 348: 552-554). Thus even when enrichments are low (Marks et al. (1991) J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (several hundred) need to be analyzed for binding to antigen.

Human antibodies can be produced without prior immunization by displaying very large and diverse V-gene repertoires on phage (Marks et al. (1991) J. Mol. Biol. 222: 581-597). In one embodiment natural V_{H} and V_{L} repertoires present in human peripheral blood lymphocytes are were isolated from unimmunized donors by PCR. The V-gene repertoires were spliced together at random using PCR to create a scFv gene repertoire which is was cloned into a phage vector to create a library of 30 million phage antibodies (Id.). From this single “naive” phage antibody library, binding antibody fragments have been isolated against more than 17 different antigens, including haptens, polysaccharides and proteins (Marks et al. (1991) J. Mol. Biol. 222: 581-597; Marks et al. (1993). Bio/Technology. 10: 779-783; Griffiths et al. (1993) EMBO J. 12: 725-734; Clackson et al. (1991) Nature. 352: 624-628). Antibodies have been produced against self proteins, including human thyroglobulin, immunoglobulin, tumor necrosis factor and CEA (Griffiths et al. (1993) EMBO J. 12: 725-734). It is also possible to isolate antibodies against cell surface antigens by selecting directly on intact cells. The antibody fragments are highly specific for the antigen used for selection and have affinities in the 1:M to 100 nM range (Marks et al. (1991) J. Mol. Biol. 222: 581-597; Griffiths et al. (1993) EMBO J. 12: 725-734). Larger phage antibody libraries result in the isolation of more antibodies of higher binding affinity to a greater proportion of antigens.

It will also be recognized that antibodies can be prepared by any of a number of commercial services (e.g., Berkeley antibody laboratories, Bethyl Laboratories, Anawa, Eurogenetec, etc.).

C) Assay Optimization.

The assays of this invention have immediate utility in screening for agents that modulate the NELL-1 expression of a cell, tissue or organism. The assays of this invention can be optimized for use in particular contexts, depending, for example, on the source and/or nature of the biological sample and/or the particular test agents, and/or the analytic facilities available. Thus, for example, optimization can involve determining optimal conditions for binding assays, optimum sample processing conditions (e.g. preferred PCR conditions), hybridization conditions that maximize signal to noise, protocols that improve throughput, etc. In addition, assay formats can be selected and/or optimized according to the availability of equipment and/or reagents. Thus, for example, where commercial antibodies or ELISA kits are available it may be desired to assay protein concentration. Conversely, where it is desired to screen for modulators that alter transcription the NELL-1 gene, nucleic acid based assays are preferred.

Routine selection and optimization of assay formats is well known to those of ordinary skill in the art.

II. Pre-Screening for Agents that Bind NELL-1

In certain embodiments it is desired to pre-screen test agents for the ability to interact with (e.g. specifically bind to) a NELL-1 nucleic acid or polypeptide. Specifically binding test agents are more likely to interact with and thereby modulate NELL-1 expression and/or activity. Thus, in some preferred embodiments, the test agent(s) are pre-screened for binding to NELL-1 nucleic acids or to NELL-1 proteins before performing the more complex assays described above.

In one embodiment, such pre-screening is accomplished with simple binding assays. Means of assaying for specific binding or the binding affinity of a particular ligand for a nucleic acid or for a protein are well known to those of skill in the art. In preferred binding assays, the NELL-1 protein or nucleic acid is immobilized and exposed to a test agent (which can be labeled), or alternatively, the test agent(s) are immobilized and exposed to an NELL-1 protein or to a NELL-1 nucleic acid (which can be labeled). The immobilized moiety is then washed to remove any unbound material and the bound test agent or bound NELL-1 nucleic acid or protein is detected (e.g. by detection of a label attached to the bound molecule). The amount of immobilized label is proportional to the degree of binding between the NELL-1 protein or nucleic acid and the test agent.

III. Agents for Screening.

While, in one embodiment, the assays described above provided methods of detecting the presence or absence, or quantifying expression of NELL-1, it will be appreciated that the same assays can be used to screen for agents that modulate the expression of and/or the activity of an MT-SP1 serine protease. To screen for potential modulators, the assays described above are performed in the presence of one or more test agents or are performed using biological samples from cells and/or tissues and/or organs and/or organisms exposed to one or more test agents. The MT-SP1 activity and/or expression level is determined and, in a preferred embodiment, compared to the activity level(s) observed in “control” assays (e.g., the same assays lacking the test agent). A difference between the MT-SP1 expression and/or activity in the “test” assay as compared to the control assay indicates that the test agent is a “modulator” of SP1 expression and/or activity.

In a preferred embodiment, the assays of this invention level are deemed to show a positive result, e.g. elevated expression and/or MT-SP1 activity, genes, when the measured protein or nucleic acid level or protein activity is greater than the level measured or known for a control sample (e.g. either a level known or measured for a normal healthy cell, tissue or organism mammal of the same species not exposed to the or putative modulator (test agent), or a “baseline/reference” level determined at a different tissue and/or a different time for the same individual). In a particularly preferred embodiment, the assay is deemed to show a positive result when the difference between sample and “control” is statistically significant (e.g. at the 85% or greater, preferably at the 90% or greater, more preferably at the 95% or greater and most preferably at the 98% or greater confidence level).

IV. High Throughput Screening.

The assays of this invention are also amenable to “high-throughput” modalities. Conventionally, new chemical entities with useful properties (e.g., modulation of NELL-1 expression or activity) are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A) Combinatorial Chemical Libraries

Recently, attention has focused on the use of combinatorial chemical libraries to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, 26 Dec. 1991), encoded peptides (PCT Publication WO 93/20242, 14 Oct. 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

B) High Throughput Assays of Chemical Libraries.

Any of the assays for that modulate expression of NELL-1 or that alter the binding specificity and/or activity of NELL-1 polypeptides are amenable to high throughput screening. As described above, having determined that NELL-1 expression is associated with bone mineralization, likely modulators either inhibit or increase bone mineralization. Preferred assays thus detect inhibition of transcription (i.e., inhibition of mRNA production) by the test compound(s), inhibition of protein expression by the test compound(s), or binding to the gene (e.g., gDNA, or cDNA) or gene product (e.g., mRNA or expressed protein) by the test compound(s). Alternatively, the assay can detect inhibition of the characteristic activity of the NELL-1 polypeptide.

High throughput assays for the presence, absence, or quantification of particular nucleic acids or protein products are well known to those of skill in the art. Similarly, binding assays are similarly well known. Thus, for example, U.S. Pat. No. 5,559,410 discloses high throughput screening methods for proteins, U.S. Pat. No. 5,585,639 discloses high throughput screening methods for nucleic acid binding (ie., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

V. Increasing Bone Mineralization Using NELL-1 Nucleic Acids and/or Polypeptides.

In still another embodiment, this invention provides methods and compositions to enhance bone growth. This is useful in a variety of contexts including, but not limited to, bone reconstruction, such as is used to reconstruct defects occurring as a result of trauma, cancer surgery or errors in development the treatment of osteogenesis imperfecta, the treatment of osteoporosis, and the healing of major or minor bone fractures.

The methods generally involve increasing NELL-1 protein concentration at or near a bone or at or in a bone progenitor cell and/or contacting a cell (e.g. a bone progenitor cell) with a NELL-1 polypeptide or with a vector encoding a NELL-1 polypeptide. This can be accomplished by transforming a bone precursor cell so that it expresses elevated levels of endogenous NELL-1 or so that it expresses NELL-1 from an exogenous transfected NELL-1 nucleic acid, or by contacting the bone, bone fracture site, bone precursor cells with NELL-1 protein(s) or local or systemic administration of a NELL-1 protein.

As used herein, the term “bone progenitor cells” refers to any or all of those cells that have the capacity to ultimately form, or contribute to the formation of, new bone tissue. This includes various cells in different stages of differentiation, such as, for example, stem cells, macrophages, fibroblasts, vascular cells, osteoblasts, chondroblasts, osteoclasts, and the like. Bone progenitor cells also include cells that have been isolated and manipulated in vitro, e.g., subjected to stimulation with agents such as cytokines or growth factors or even genetically engineered cells. The particular type or types of bone progenitor cells that are stimulated using the methods and compositions of the invention are not important, so long as the cells are stimulated in such a way that they are activated and, in the context of in vivo embodiments, ultimately give rise to new bone tissue.

The term “bone progenitor cell” is also used to particularly refer to those cells that are located within, are in contact with, or migrate towards (i.e., “home to”), bone progenitor tissue and which cells directly or indirectly stimulate the formation of mature bone. As such, the progenitor cells may be cells that ultimately differentiate into mature bone cells themselves, i.e., cells that “directly” form new bone tissue. Cells that, upon stimulation, attract further progenitor cells or promote nearby cells to differentiate into bone-forming cells (e.g., into osteoblasts, osteocytes and/or osteoclasts) are also considered to be progenitor cells in the context of this disclosure-as their stimulation “indirectly” leads to bone repair or regeneration. Cells affecting bone formation indirectly may do so by the elaboration of various growth factors or cytokines, or by their physical interaction with other cell types. The direct or indirect mechanisms by which progenitor cells stimulate bone repair is not necessary a consideration in practicing this invention. Bone progenitor cells and bone progenitor tissues may be cells and tissues that, in their natural environment, arrive at an area of active bone growth, repair or regeneration. In terms of bone progenitor cells, these may also be cells that are attracted or recruited to such an area. These may be cells that are present within an artificially-created osteotomy site in an animal model. Bone progenitor cells may also be isolated from animal or human tissues and maintained in an in vitro environment. Suitable areas of the body from which to obtain bone progenitor cells are areas such as the bone tissue and fluid surrounding a fracture or other skeletal defect (whether or not this is an artificially created site), or indeed, from the bone marrow. Isolated cells may be stimulated using the methods and compositions disclosed herein and, if desired, be returned to an appropriate site in an animal where bone repair is to be stimulated. In such cases, the nucleic-acid containing cells would themselves be a form of therapeutic agent. Such ex vivo protocols are well known to those of skill in the art. In preferred embodiments of the invention, the bone progenitor cells and tissues will be those cells and tissues that arrive at the area of bone fracture or damage that one desires to treat. Accordingly, in treatment embodiments, there is no difficulty associated with the identification of suitable target progenitor cells to which the present therapeutic compositions should be applied. It is sufficient in such cases to obtain an appropriate stimulatory composition (e.g. a NELL-1 polypeptide), as disclosed herein, and contact the site of the bone fracture or defect with the composition. The nature of this biological environment is such that the appropriate cells will become activated in the absence of any further targeting or cellular identification by the practitioner.

A) Transformation of Cells to Increase NELL-1 Production.

In a more preferred embodiment, the NELL-1 expressing nucleic acids (e.g., cDNA(s)) are cloned into gene therapy vectors that are competent to transfect cells (such as human or other mammalian cells) in vitro and/or in vivo.

Several approaches for introducing nucleic acids into cells in vivo, ex vivo and in vitro have been used. These include lipid or liposome based gene delivery (WO 96/18372; WO 93/24640; Mannino and Gould-Fogerite (1988) BioTechniques 6(7): 682-691; Rose U.S. Pat No. 5,279,833; WO 91/06309; and Felgner et al. (1987) Proc. Natl. Acad. Sci. USA 84: 7413-7414) and replication-defective retroviral vectors harboring a therapeutic polynucleotide sequence as part of the retroviral genome (see, e.g., Miller et al. (1990) Mol. Cell. Biol. 10:4239 (1990); Kolberg (1992) J. NIH Res. 4: 43, and Cornetta et al. (1991) Hum. Gene Ther. 2: 215).

For a review of gene therapy procedures, see, e.g., Anderson, Science (1992) 256: 808-813; Nabel and Felgner (1993) TIBTECH 11: 211-217; Mitani and Caskey (1993) TIBTECH 11: 162-166; Mulligan (1993) Science, 926-932; Dillon (1993) TIBTECH 11: 167-175; Miller (1992) Nature 357: 455-460; Van Brunt (1988) Biotechnology 6(10): 1149-1154; Vigne (1995) Restorative Neurology and Neuroscience 8: 35-36; Kremer and Perricaudet (1995) British Medical Bulletin 51(1) 31-44; Haddada et al. (1995) in Current Topics in Microbiology and Immunology, Doerfler and Böhm (eds) Springer-Verlag, Heidelberg Germany; and Yu et al., (1994) Gene Therapy, 1:13-26.

Widely used retroviral vectors include those based upon murine leukemia virus (MuLV), gibbon ape leukemia virus (GaLV), Simian Immuno deficiency virus (SIV), human immuno deficiency virus (HIV), and combinations thereof. See, e.g., Buchscher et al. (1992) J. Virol. 66(5) 2731-2739; Johann et al. (1992) J. Virol. 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al., Gene Therapy (1994) supra). The vectors are optionally pseudotyped to extend the host range of the vector to cells which are not infected by the retrovirus corresponding to the vector. The vesicular stomatitis virus envelope glycoprotein (VSV-G) has been used to construct VSV-G-pseudotyped HIV vectors which can infect hematopoietic stem cells (Naldini et al. (1996) Science 272:263, and Akkina et al. (1996) J Virol 70:2581).

Adeno-associated virus (AAV)-based vectors are also used to transduce cells with target nucleic acids, e.g., in the in vitro production of nucleic acids and peptides, and in in vivo and ex vivo gene therapy procedures. See, West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invest. 94:1351 for an overview of AAV vectors. Construction of recombinant AAV vectors are described in a number of publications, including Lebkowski, U.S. Pat. No. 5,173,414; Tratschin et al. (1985) Mol. Cell. Biol. 5(11):3251-3260; Tratschin, et al. (1984) Mol. Cell. Biol., 4: 2072-2081; Hermonat and Muzyczka (1984) Proc. Natl. Acad. Sci. USA, 81: 6466-6470; McLaughlin et al. (1988) and Samulski et al. (1989) J. Virol., 63:03822-3828. Cell lines that can be transformed by rAAV include those described in Lebkowski et al. (1988) Mol. Cell. Biol., 8:3988-3996. Other suitable viral vectors include herpes virus and vaccinia virus.

U.S. Pat. Nos. 5,942,496 and 5,763,416 disclose methods, compositions, kits and devices for use in transferring nucleic acids into bone cells in situ and/or for stimulating bone progenitor cells (see also, Evans and Robbins (1995) J. Bone and Joint Surgery, 77-A(7):1103-1114, Wolff et al. (1992) J. Cell Sci., 103:1249-1259).

B) Administration of Exogenously Produced NELL-1.

1) Delivery of NELL-1 Proteins to Target Cells.

The NELL-1 proteins (or biologically active fragments thereof) of this invention are useful for intravenous, parenteral, topical, oral, or local administration (e.g., by aerosol or transdermally). Particularly preferred modes of administration include intra-arterial injection, injection into fracture sites or delivery in a biodegradable matrix. The NELL-1 proteins agents are typically combined with a pharmaceutically acceptable carrier (excipient) to form a pharmacological composition. Pharmaceutically acceptable carriers can contain a physiologically acceptable compound that acts, for example, to stabilize the composition or to increase or decrease the absorption of the agent. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the anti-mitotic agents, or excipients or other stabilizers and/or buffers.

Other physiologically acceptable compounds include wetting agents, emulsifying agents, dispersing agents or preservatives which are particularly useful for preventing the growth or action of microorganisms. Various preservatives are well known and include, for example, phenol and ascorbic acid. One skilled in the art would appreciate that the choice of a pharmaceutically acceptable carrier, including a physiologically acceptable compound depends, for example, on the rout of administration of the anti-mitotic agent and on the particular physio-chemical characteristics of the anti-mitotic agent. Preferred formulations for the delivery of bone morphogenic proteins (BMPs) are described in detail in U.S. Pat. No. 5,385,887.

The pharmaceutical compositions can be administered in a variety of unit dosage forms depending upon the method of administration. For example, unit dosage forms suitable for oral administration include powder, tablets, pills, capsules and lozenges. It is recognized that the NELL-1 protein(s), if administered orally, must be protected from digestion. This is typically accomplished either by complexing the protein with a composition to render it resistant to acidic and enzymatic hydrolysis or by packaging the protein in an appropriately resistant carrier such as a liposome. Means of protecting compounds from digestion are well known in the art (see, e.g., U.S. Pat. No. 5,391,377 describing lipid compositions for oral delivery of therapeutic agents).

The pharmaceutical compositions of this invention are particularly useful for topical administration e.g., in surgical wounds to treat facilitate bone reconstruction and/or repair. In another embodiment, the compositions are useful for parenteral administration, such as intravenous administration or administration into a body cavity or lumen of an organ. The compositions for administration will commonly comprise a solution of the NELL-1 protein dissolved in a pharmaceutically acceptable carrier, preferably an aqueous carrier for water-soluble proteins. A variety of carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

The concentration of NELL-1 protein in these formulations can vary widely, and will be selected primarily based on fluid volumes, viscosities, body weight and the like in accordance with the particular mode of administration selected and the patient's needs. Typically the NELL-1 proteins are utilized in the form of a pharmaceutically acceptable solution (including reconstitution from a lyophilized form). It is optimal to solubilize the osteogenic protein at concentrations of at least about 1 mg/ml, preferably about 2 to 8 mg/ml, so that a pharmaceutically effective amount of protein can be delivered without undue volumes of carrier being necessary. For some applications, concentrations above 2 mg/ml may be desirable.

As alluded to above, the dosage regimen will be determined by the clinical indication being addressed, as well as by various patient variables (e.g. weight, age, sex) and clinical presentation (e.g. extent of injury, site of injury, etc.). In general, the dosage of osteogenic protein will be in the range of from 1 to about 10000 ηg, preferably from about about 10 to 1000 μg, more preferably from about 10 to 100 μg.

2) Bone Graft Materials.

Bone wounds, as well as many other wound models, initiate a release of biologically active agents critical to the wound healing process. Bone morphogenic proteins (BMP), which naturally occur in bone, once released from the wound, stimulate osteoinduction and regenerate lost or damaged bone tissue. These same proteins, in a purified form, can be used to stimulate bone growth into a biodegradable matrix allowing for artificial creation of bone both within and external to the normal skeletal boundaries. Without being bound to a particular theory, it is believed that NELL-1 proteins can be used to stimulate bone re-mineralization in a manner analogous to the use of bone morphogenic proteins.

NELL-1 proteins can be administered systemically as discussed above. In addition, or alternatively, the NELL-1 protein can be applied directly to a bone or bone fracture site. This can be accomplished during surgery (e.g. when setting complex fractures, when reconstructing bone, when performing bone transplants, etc.) or can be accomplished by direct injection.

In certain preferred embodiments, particularly where bone reconstruction or repair is performed surgically, it is desired to administer the NELL-1 protein using a sustained delivery “vehicle”. Sustained delivery vehicles include, but are not limited to biodegradable delivery vehicles. Preferred biodegradable delivery vehicles are preferably porous.

Much work has been done in developing biodegradable porous delivery vehicles for the controlled release of substances while also providing a location for cellular attachment and guided tissue regeneration. Biodegradable materials often separated into two categories: 1) those which are hydrophilic; and 2) those which are hydrophobic. Hydrophilic materials (demineralized freeze dried bone, ceramic, fibrin, gelatin, etc.) possess a high affinity for water which provides for easy incorporation of aqueous NELL-1 protein solutions within the internal porosity of the material. Hydrophobic materials (L-polylactic acid, D,L-polylactic acid, poly-glycolic acid, etc.), while potentially limitless in their range of porosities, gross size, shape and mechanical characteristics are more difficult to “infiltrate” with aqueous solutions. To increase deposition of solutions into internal surfaces of such materials, hydrophobic materials are often impregnated with the protein or a surfactant is used to facilitate impregnation with the protein (e.g. NELL-1).

Detailed descriptions of various biodegradable delivery materials comprising materials such as fibrinogen, polylactic acid, porous ceramics, gelatin, agar, and the like, can be found, e.g., in U.S. Pat. Nos: 5,736,160, 4,181,983, 4,186,448, 3,902,497, 4,442,655, 4,563,489, 4,596,574, 4,609,551, 4,620,327, and 5,041,138.

Other delivery vehicles include, but are not limited to bone graft materials. Bone graft materials can be derived from natural materials (e.g. transplanted bone or bone fragments), synthetic materials (e.g. various polymers and/or ceramics) or combinations of both. Bone graft materials are typically utilized to fill voids or otherwise replace lost bone material. Such graft materials are also often provided as components of prosthetic devices (e.g., bone replacements or supports) to facilitate tight bondinglannealing of the prosthetic with the living bone.

Bone grafts using bioactive glasses and calcium phosphates, collagen, mixtures and the like have good biocompatibility and give rise to bone tissue formation and incorporation in some cases. A number of different glasses, glass-ceramics, and crystalline phase materials have been used, either alone or in combination with acrylic polymerizable species, and other families of polymers, for restorative purposes. These include hydroxyapatite, fluorapatite, oxyapatite, Wollastonite, anorthite, calcium fluoride, agrellite, devitrite, canasite, phlogopite, monetite, brushite, octocalcium phosphate, Whitlockite, tetracalcium phosphate, cordierite, and Berlinite. Representative patents describing such uses include U.S. Pat. Nos. 3,981,736, 4,652,534, 4,643,982, 4,775,646, 5,236,458, 2,920,971, 5,336,642, and 2,920,971. Additional references include Japanese Patent No. 87-010939 and German Patent OS 2,208,236. Other references may be found in W. F. Brown, “Solubilities of Phosphate & Other Sparingly Soluble Compounds,” Environmental Phosphorous Handbook, Ch. 10 (1973). In addition to the foregoing, certain animal derived materials, including coral and nacre, have also been used in biomaterials for restorative purposes.

Other bone graft materials include a pliable, moldable acrylic-based bone cement reinforced with from 15 to 75% by weight of a bioactive glass together with between 1 and 10% by weight of vitreous mineral fibers (U.S. Pat. No. 4,239,113), bone fillers such as tricalcium phosphate and bioceramic A₂ into bisphenol-A-diglycidyl methacrylate (bis GMA) polymerizable through the action of peroxide systems such as benzoyl peroxide mixed with amines, (Vuillemin et al. (1987) Arch. Otolygol. Head Neck Surg. 113: 836-840). Two component, resin composites containing both salicylates and acrylates, cured through a calcium hydroxide cement reaction are described in U.S. Pat. No. 4,886,843, while U.S. Pat. Nos. 5,145,520 and 5,238,491, discloses fillers and cements. The foregoing materials can be fabricated so as to incorporate NELL-1 proteins.

In addition, graft materials that include bone morphogenic proteins are known. Thus, for example, U.S. Pat. No. 4,394,370 describes complexes of reconstituted collagen and demineralized bone particles or reconstituted collagen and a solubilized bone morphogenetic protein fabricated in a sponge suitable for in vivo implantation in osseous defects are disclosed. Similarly U.S. Pat. No. 5,824,084 describes substrates made from a biocompatible, implantable graft material, preferably having a charged surface. Examples of biocompatible, implantable graft materials include synthetic ceramics comprising calcium phosphate, some polymers, demineralized bone matrix, or mineralized bone matrix. These materials may additionally contain cell adhesion molecules bound to the surface of the substrate. The term “cell adhesion molecules” refers collectively to laminins, fibronectin, vitronectin, vascular cell adhesion molecules (V-CAM) and intercellular adhesion molecules (I-CAM) and collagen. Particularly suitable graft materials include, for example, isolated mineralized cancerous bone sections, powders or granules of mineralized bone, demineralized cancellous bone sections, powders or granules of demineralized bone, guanidine-HCl extracted demineralized bone matrix, sintered cortical or cancellous bone, coralline hydroxyapatite sold by Interpore under the trade name Interpore 500, and granular ceramics such as that incorporated into the bone graft substitute Collagraft sold by Zimmer, or filamentous sponges such as those made from collagen by Orquest. NELL-1 proteins can be incorporated into any of these graft materials or substituted in place of the bone morphogenic protein.

VII. Kits.

In still another embodiment, this invention provides kits for practice of the assays or use of the compositions described herein. In one preferred embodiment, the kits comprise one or more containers containing antibodies and/or nucleic acid probes and/or substrates suitable for detection of NELL-1 expression and/or activity levels. The kits may optionally include any reagents and/or apparatus to facilitate practice of the assays described herein. Such reagents include, but are not limited to buffers, labels, labeled antibodies, labeled nucleic acids, filter sets for visualization of fluorescent labels, blotting membranes, and the like.

In another embodiment, the kits can comprise a container containing a NELL-1 protein, or a vector encoding a NELL-1 protein and/or a cell comprising a vector encoding a NELL-1 protein.

In addition, the kits may include instructional materials containing directions (i.e., protocols) for the practice of the assay methods of this invention or the administration of the compositions described here along with counterindications. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### NELL-1 Enhances Mineralization in Fetal Calvarial Osteoblastic Cells

The nucleotide sequence of the full length cDNA of the NELL-1 gene described herein has approximately 61% homology to the chicken Nel gene, and therefore, was named human NELL-1 (Watanabe et al. (1996) Genomics. 38(3),273-276). NELL-1 proteins contain a signal peptide, a NH₂-terminal thrombospondin (TSP)-like module (François and Bier (1995) Cell. 80(1):19-20), five von Willebrand factor C domains, and six EGF-like domains.

The human NELL-1 gene expressions were primarily localized in the mesenchymal and osteoblast cells at the osteogenic front, along the parasutural bone margins, and within the condensing mesenchymal cells of newly formed bone. A human multiple-organ tissue mRNA blot showed that human NELL-1 was specifically expressed in fetal brain but not in fetal lung, kidney or liver. We also demonstrated that NELL-1 was expressed in rat calvarial osteoprogenitor cells but was largely absent in rat tibia, stromal cell, and fibroblast cell culture. Our data suggest that the NELL-1 gene is preferentially expressed in cranial intramembranous bone and neural tissue (neural crest origin).

A) Materials and Methods.

Whole mouse embryo RNA analysis from the fetal gestation day 7, 11, 14, 17, was performed. Adenoviruses (AD5 with an E1-A knock-out and MCV promoter) carrying NELL-1 cDNA were constructed and infected into rat fetal calvarial primary cell cultures and MC3T3 cell lines. Viruses were constructed according the following protocol: the 293 cells were co-transfected with 10 mg each of pJM17 (containing defective adenovirus genome) and pAC-CMV-based plasmid (containing sense or antisense rat NELL-1 using CaPO₄) to produce recombinant adenovirus vectors expressing rat NELL-1 in 10-14 days. Viruses were plaque-purified and Southern blots were performed to assure the incorporation of the NELL-1 gene. Adenoviruses containing the β-Galactosidase gene were used as a control and examined for the efficacy of infection with different cell types. Approximately 80-90% infection efficiency was observed in both MC3T3 and NIH3T3 cells.

Von Kossa staining was performed on 14, 17, 21 day post-infections. Area of mineralization was quantitated by ImagePro system. Statistical analysis was performed by two-tailed Student's t test. A statistical P value of *p<0.01 was considered significant. RNA from cells over-expressing NELL-1 was extracted and mouse cDNA array analysis was performed. Hybridization signals were quantitated by phosphoimager.

B) Results.

NELL-1 mRNA was faintly expressed from day 14 of gestation with mild increase over the gestation period. Day 14 gestation is the time point when fetal calvaria starts to mineralize. Both primary rat fetal calvarial cell cultures and MC3T3 cell cultures over-expressing NELL-1 showed an increase in mineralization over the β-Galactosidase control. Over-expression of NELL-1 enhanced mineralization in calvarial osteogenic primary cell cultures by approximately 30 folds on day 17 post-infection compared to the control. These results were based on Von Kossa staining and quantitated by ImagePro software. This relative increase decreased to 2 fold by day 21 post infection. Mouse cDNA array results from NELL-1 infected MC3T3 cells showed 20% down regulation in BMP-7 gene expression and a three fold up regulation of the Split Hand and Foot gene compared to the control. These two genes are closed related to bone formation and craniofacial development.

C) Discussions and Conclusions.

In this study, we clearly confirmed that NELL-1 is closely associated with bone formation and it enhanced mineralization of the calvarial osteoblast-like cells. Some of the down stream effectors identified clearly play important roles in bone formation and embryological development. Premature cranial suture closure, as seen in CS, may be due to overproduction of cranial bone, and therefore, possibly be associated with the over-expression of the NELL-1 molecule. These results and the preliminary protein function analysis results of the NELL-1 classify this protein as a biologically relevant molecule. As a possible role of NELL-1, these proteins may act as a modulator, interacting with other growth factors. Recently, TSP-1 was shown to be a major activator of TGFβ-1 (François and Bier (1995) Cell. 80(1):19-20 ). TGFβ-1 is secreted by most cells in an inactive form that is unable to interact with cellular receptors. The activity of TGFβ-1 is initially masked by its noncovalent association with a dimer of its NH₂-terminal propeptide, called latency-associated protein (LAP). In activating TGFβ-1 extracellularly, TSP-1 interacts with the NH₂-terminal region of LAP, forming a trimolecular complex. Within the complex, a conformational change takes place that makes TGFβ-1 accessible to the receptor. Molecules with high homology like chordin, which possess four vWF C domains (presumably homotrimer), is secreted during gastrulation and plays a pivotal role in the Xenopus dorsoventral patterning (Crawford et al. (1998) Cell. 93(7):1159-1170). Recently, chordin was revealed to directly bind to ventral BMP-4 (bone morphogenetic proteins 4, one of the TGFβ superfamily) and neutralize the BMP-4 activity (Piccolo et al. (1996) Cell, 86(4):589-598). These results suggest that NELL-1 protein may execute their unidentified functions extracellularly by interacting with some of the TGFβ superfamily members. Since TGFβ-1 is known as a regulator of osteogenesis, NELL-1's effect in enhancing mineralization may be related to its interaction with the TGFβ superfamily.

### Example 2

### Craniosynostosis in Transgenic Mice Overexpressing Nell-1

Previously, we reported Nell-1 as a novel molecule overexpressed during premature cranial suture closure in patients with craniosynostosis (CS), one of the most common congenital craniofacial deformities. Here we describe the creation and analysis of transgenic mice overexpressing Nell-1. Nell-1 transgenic animals exhibited CS-like phenotypes that ranged from simple to compound synostoses. Histologically, the osteogenic fronts of abnormally closing/closed sutures in these animals revealed calvarial overgrowth and overlap along with increased osteoblast differentiation and reduced cell proliferation. Furthermore, anomalies were restricted to calvarial bone, despite generalized, non-tissue-specific overexpression of Nell-1. In vitro, Nell-1 overexpression accelerated calvarial osteoblast differentiation and mineralization under normal culture conditions. Moreover, Nell-1 overexpression in osteoblasts was sufficient to promote alkaline phosphatase expression and micronodule formation. Conversely, downregulation of Nell-1 inhibited osteoblast differentiation in vitro. In summary, Nell-1 overexpression induced calvarial overgrowth resulting in premature suture closure in a rodent model. Nell-1, therefore, has a novel role in CS development, perhaps as part of a complex chain of events resulting in premature suture closure. On a cellular level, Nell-1 expression may modulate and be both sufficient and required for osteoblast differentiation.

Methods

Preparation of Transgenic Mice Overexpressing Nell-1.

Rat Nell-1 cDNA was subcloned from pTM-70 (13, 14) into pCDNA1.1 (Invitrogen, Carlsbad, Calif., USA), which uses a CMV promoter and an SV40 polyadenylation site. The recombinant plasmid was first transfected into MC3T3 cells (a mouse calvarial cell line) to verify proper protein expression (data not shown). The 4.76-kb DNA fragment containing the CMV promoter, Nell-1 cDNA, and the SV40 polyadenylation site was then used for microinjection of oocytes. B6C3 mice were used to generate transgenic mice using standard protocols (15). The founders were mated with their nontransgenic littermates to set up transgenic lines.

Analysis of Transgene Copy Number.

Transgene copy numbers were estimated by PCR and Southern blot analysis. The PCR protocol of establishing transgene copy number was obtained at http://www.med.umich.edu/tamc/spike.html (16). The mass of transgene DNA per 5 □ g genomic DNA was calculated as N bp transgene DNA/3 [symbol] 109 genomic DNA, based on the assumption that the haploid content of a mammalian genome is 3 [symbol] 109 bp and that it takes 10 □g DNA to spike. The size of the insert is 4.76 kb, and the one-copy standard is 7.933 pg per 10 □g genomic DNA. Thirty cycles of PCR were performed and products were separated on electrophoresis gels with ethidium bromide. The intensities were calculated using Eagle Eye II (Stratagene, La Jolla, Calif., USA).

Immunohistochemistry.

Detailed preparation of Nell-1 antibody has been documented by Kuroda et al. (13, 14). The antibody recognizes the COOH-terminal region of Nell-1 (CSVDLECIENN). The specificity of the antibody was confirmed by Western blot using protein extracted from Nell-1-transfected NIH3T3 cells. A standard avidinbiotin complex/immunoperoxidase protocol (Vector Elite Kit; Vector Laboratories Inc., Burlingame, Calif., USA) was used with 1:100 Nell-1 antibody dilution. Diaminobenzidine peroxidase substrate and 3-amino-9-ethylcarbazole were used for visualization, and sections were counterstained with hematoxylin.

Magnetic Resonance Imaging.

Magnetic resonance imaging (MRI) was performed on formalin-preserved specimens using a Bruker Biospec MR imager (Bruker BioSpin GmbH, Rheinstetten, Germany) with a 7.0-T, 18-cm clear-bore magnet equipped with a microimaging gradient set and a 35-mm internal diameter birdcage radiofrequency coil. Transaxial and sagittal images of the brain and calvarium were obtained using a gradient echo filtered imaging steady-state pulse sequence with the following parameters: TR/TE, 229.3/64.1 ms; flip angle, 30°; field of view, 2.3 cm; matrix, 256×256; slice thickness, 1 mm; and number of excitations, 8. Inplane spatial resolution was approximately 90 μm.

Microcomputerized Tomography Scan.

All the data were collected at 30 kVp and 750 mA. The data was reconstructed using the cone-beam algorithm supplied with the MicroCat scanner (Oak Ridge National Laboratory, Oak Ridge, Tenn., USA). The matrix was 256×256×256, yielding an isotropic resolution of 140 μm. The quantitative procedures involve the placement of bone phantoms (long rods in the images) containing 0, 50, 250, and 750 mg/cc hydroxyapatite. Visualization of the data was performed using MetaMorph (two dimensional) (Universal Imaging Corp., West Chester, Penn., USA) and Amira (three dimensional) (Indeed—Visual Concepts GmbH, Berlin, Germany).

In vivo Proliferation Analysis.

Newborn mice were injected with BrdU at 100 μg/g. Animals were sacrificed 2 hours after injection. The animals were fixed and immunostained with BrdU antibodies (Sigma-Aldrich, St. Louis, Mo., USA). Calvarial sutures, brain, and tibiae from transgenic animals and their normal littermates were compared.

Recombinant Defective Adenovirus Vectors Harboring Nell-1 (AdNell-1) and Antisense Nell-1 (AdAntiNell-1).

Rat Nell-1 cDNA was inserted bidirectionally between the human CMV IE1 promoter and the SV40 splice/polyadenylation site flanked by nucleotide sequences from 1 to 454 and from 3,334 to 6,231 of the AdS virus. The resulting plasmid, pAdCMV-Nell-1, transcribes Nell-1 leftward relative to the standard Ad5 map. The recombinant adenovirus (Ad) (AdNell-1) were isolated by cotransfecting 293 cells with pAdCMV-Nell-1 and pJM17 (Microbix Biosystems Inc., Toronto, Canada), resulting in vectors defective in the E1-A viral gene. Clones of recombinant virus were plaque purified and confirmed by Southern blot analysis. Both AdNell-1 and AdLacZ were grown to a high titer and purified once through a CsCl cushion and again on a continuous CsCl gradient. The resulting stocks were 5×109 pfu/ml as assayed by plaque formation on 293 cells. Northern and Western blots were performed to assure the incorporation and expression of the Nell-1 gene and its protein product.

Rat Calvarial Primary Cell Cultures (FRCCs).

The isolation of osteogenic cells from embryonic day 18 (E18) rat calvaria was performed as previously described (12). The cells collected from digestions four, five, and six were pooled and plated at 2.5×104/cm2. Cells within passage two were used. Adenoviral infection of osteoblasts. In order to observe the effects of overexpressing Nell-1, osteoblasts from different lineages were grown to 80% confluence in six-well plates. The media was aspirated and an infective dose (20 pfu/cell in 1 ml serum-free medium) was added to the cultures. Five sets of AdNell-1, AdAntiNell-1, and control Ad carrying β-Galactosidase (Adβ-Gal) were used. On days 12, 15, and 21 after infection, von Kossa staining was performed. The percentage of area mineralized was analyzed using the Image-Pro Plus system (Media Cybernetics, Silver Spring, Md., USA). Comparisons between mice were made using the Student t test. In order to observe the effects of downregulating Nell-1, AdAntiNell-1 was added to fetal rat calvarial cell (FRCC) cultures as described above.

Microarray Analysis.

Microarrays were performed using RNA from AdNell-1- and Ad β-Gal-infected MC3T3 cells at 6, 9, and 12 days after infection. I. Nishimura and the University of California Los Angeles Microarrays Core Facility staff have developed bone-related microarrays. The microarrays contain over 37 genes with more than ten internal control genes. Confirmed markers include the following: bone matrix proteins (osteopontin, osteonectin, osteocalcin, bone sialoprotein); receptors (α2-integrin, vitamin D receptor, parathyroid receptor, estrogen receptor); osteoblastic markers (alkaline phosphatase, Cbfa1); adhesive proteins (fibronectin, chondroitin sulfate proteoglycan 1, decorin, tenascin, syndecan, laminin); metalloproteinases (matrix metalloproteinases 1 and 2); growth factors (Bmp2, Bmp7); fibrillar collagens (collagens 1A1, 1A2, 3A1, 5A2, and 11A1); other collagens (collagens 4A1, 6A1, 7A1, 10A1, and 15A1); and fibril-associated collagen with interrupted triple helices (FACITs) (collagens 9A1, 9A2, 12, 14, 16, and 19). RNA (30 μg total RNA for Cy3 and 60 μg for CyS) was labeled with random hexamer primers and Cy3- or Cy5-dUTP. The reverse transcriptase-labeled probes were hybridized onto the arrays. Multiple laser scans were performed with a 418 Array Scanner (Affymetrix Inc., Santa Clara, Calif., USA) to provide mean readouts and standard deviations to verify the reproducibility of the measurements. An average of all the internal controls was calculated and used to normalize hybridization intensities using the IPLab version 3.2 MicroArray suite (Scanalytics Inc., Fairfax, Va., USA). The correlation of all osteoblastic markers as a group was calculated and compared between the AdNell-1-infected cells and the Ad β-Gal-infected control cells. RT-PCR. DNase-treated total RNA was used. After initial verification of gene fragment expression through high-cycle PCR, another low-cycle PCR was performed to quantify relative gene expression (12). For each candidate molecule, we determined the cycle number most likely to fall within the linear amplification range by successively reducing the number of cycles (range, 15-35 cycles). Electrophoreses were performed and hybridized with sequence-specific probes labeled wit P32. A PhosphorImager (Molecular Dynamics, Sunnyvale, Calif., USA) was used to measure the intensities. For each sample, the densitometry value was divided by the Gapdh value (performed at 20 cycles) and normalized. Primer sequences were as follows. Msx2: forward, 5′-CCT CGG TCA AGT CGG AAA ATI C-3′ (SEQ ID NO:2); reverse, 5′-TGG ACA GGT ACT GTI TCT GGC G-3′ (SEQ ID NO:3); probe, 5′-GAG CAC CGT GGA TAC AGG AG-3′ (SEQ ID NO:4) (annealing temperature, 68° C.). Cbfal: forward, 5′-CTG TGT GGC TCC TAA CAA GTG TG-3′ (SEQ ID NO:5); reverse, 5′-GGA TTC TGG CAA TCA CAA GCT GTC-3′ (SEQ ID NO:6); probe, 5′-CCT ACT CAC TGT CCG GGG AGT CCT GC-3′ (SEQ ID NO:7) (annealing temperature, 66° C.). Osteocalcin: forward, 5′-ATG AGG ACC CTC TCT CTG CTC-3′ (SEQ ID NO:8); reverse, 5′-GTG GTG CCA TAG ATG CGC TTG-3′ (SEQ ID NO:9), probe 5′-CAT GTC AAG CAG GGA GGG CA-3′ (SEQ ID NO:10), (annealing temperature, 66° C.). Osteopontin: forward, 5′-AGC AGG AAT ACT AAC TGC-3′ (SEQ ID NO:11); reverse, 5′-GAT TAT AGT GAC ACA GAC-3′ (SEQ ID NO:12) probe 5′-GCC CTG AGC TTA GTT CGT TG-3′ (SEQ ID NO:13), (annealing temperature,66° C.). Nell-1: (12).

Flow Cytometry Analysis.

Cells were seeded on 60-mm plates at 5×105 cells/plate. Cells were harvested at 24, 36, 48, and 72 hours after infection with AdNell-1 and Ad β-Gal. One million cells were used for flow cytometry, and this procedure was repeated three times. Hypotonic DNA staining buffer containing propidium iodide was added to the cells for flow cytometry.

Results

Construction of CMV Promoter/Nell-1 Transgenic Mice.

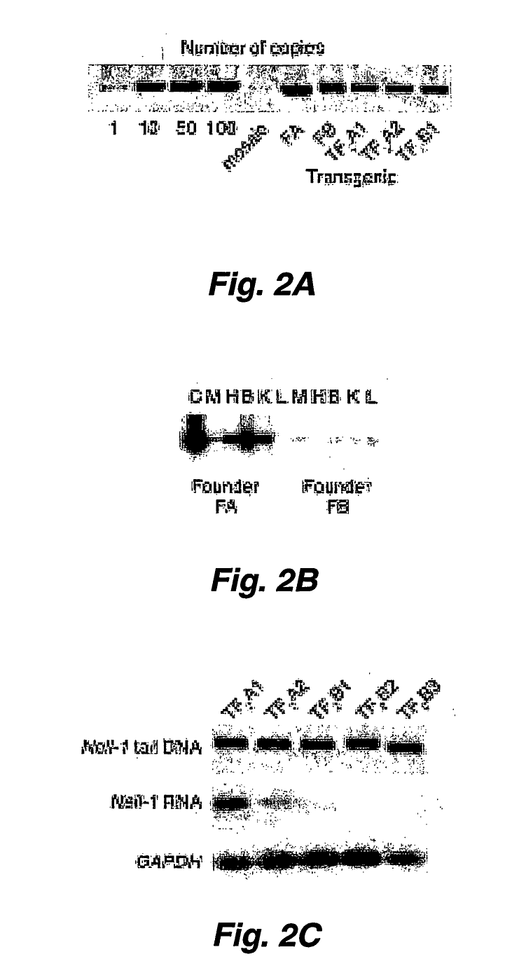

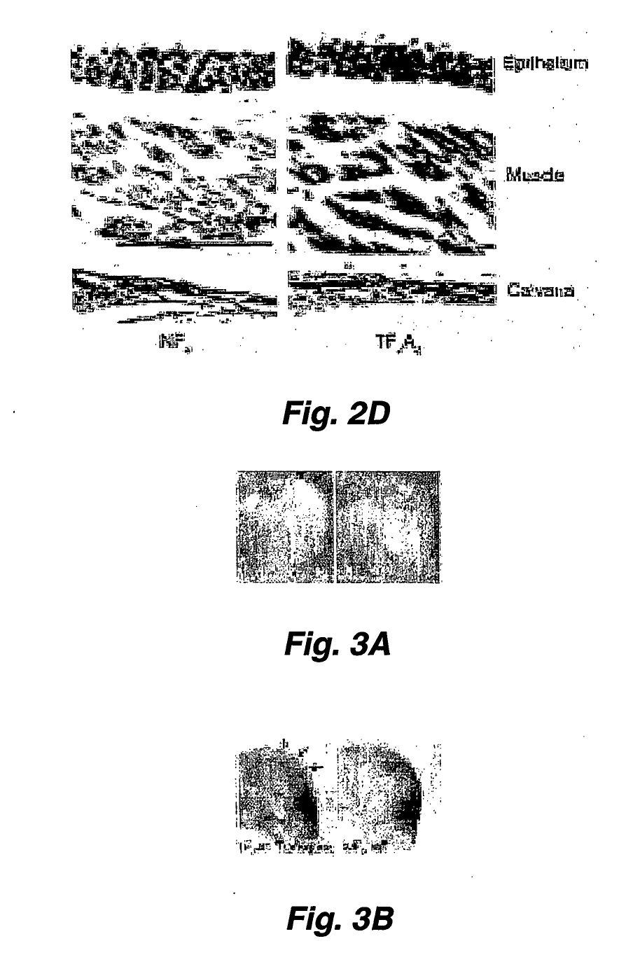

To investigate the effects of generalized Nell-1 overexpression in vivo, transgenic mice in which Nell-1 is expressed under the control of the CMV promoter were produced. Copy number was confirmed by Southern blot and PCR (FIG. 2A). RNA analysis (FIG. 2B) and immunohistochemistry (data not shown) further confirmed expression of Nell-1 in founders. Nell-1-overexpressing founders were crossed with nontransgenic littermates, and comprehensive analyses were conducted on F2 progeny. Because most human CS phenotypes are readily apparent in newborns, 42 newborn mice, representing six litters from two lines, were examined. The morphology of these mice was assessed for developmental anomalies, including suture closure. The mice were subsequently genotyped. Suture patency was determined by the absence (indicating suture closure) or the presence (indicating suture patency) of visible blood vessels underneath the suture. Suture closure was further confirmed under a dissecting microscope. Two of the six litters examined, representing 20 progeny, did not yield any newborns with obvious craniofacial defects and were Nell-1 transgene negative. These litters were not examined further. Progeny with craniofacial defects were recovered in each of the four remaining litters. The progeny of these four litters (annealing temperature, 66° C.). Osteocalcin: forward, 5′-ATG AGG ACC CTC TCT CTG CTC-3′ (SEQ ID NO:14); reverse, 5′-GTG GTG CCA TAG ATG CGC TTG-3′ (SEQ ID NO: 15), probe 5′-CAT GTC AAG CAG GGA GGG CA-3′ (SEQ ID NO:16), (annealing temperature, 66° C.). Osteopontin: forward, 5′-AGC AGG AAT ACT AAC TGC-3′ (SEQ ID NO: 17); reverse, 5′-GAT TAT AGT GAC ACA GAC-3′ (SEQ ID NO:18) probe 5′-GCC CTG AGC TTA GTT CGT TG-3′ (SEQ ID NO:19), (annealing temperature, 66° C.). Nell-1: (12). (22 mice) were analyzed further. A limitation of this rapid screening method is that mild CS with only focal points of suture closure may not be detected, and therefore Nell-1 overexpression might appear to have lower penetrance.

Thirteen (60%) of the 22 newborn progeny were transgenic, with gene copy numbers similar to the founder Nell-1 mice (prediction is 50%). Nell-1 RNA levels of the 13 Nell-1 DNA-positive transgenic F2 (TF2) mice were examined. Eight (62%) were positive for Nell-1 RNA expression. However, the level of expression varied (FIG. 2C). The reason for low or nearly absentNell-1 expression in some TF2 mice despite their high transgene copy numbers is not clear, but epigenetic effects such as heterochromatin formation around the inserts may play a significant role in the high variability of transgene expression (17). RNA levels also differed in different tissues isolated from the same litter. Liu et al. also made this observation of variegation when they overexpressed Msx2 using a CMV promoter (5, 6). Therefore transgenic Nell-1 transcription may not necessarily correlate with gene copy number, and may also vary according to cell type. To determine whether Nell-1 overexpression in our transgenic model was physiologically relevant, we compared Nell-1 RNA expression levels from the whole heads of three TF2 progeny with mild CS phenotypes to levels in nontransgenic normal littermates (NF2 mice). TF2 mice displayed up to fourfold-increased Nell-1 expression (data not shown). This was comparable to levels of NELL-1 overexpression in human CS patients in whom two- to fourfold increases have been observed (12). This suggests that Nell-1 overexpression levels in our model were clinically relevant rather than superphysiologic.

Phenotypic Analyses of Nell-1 Transgenic Mice.

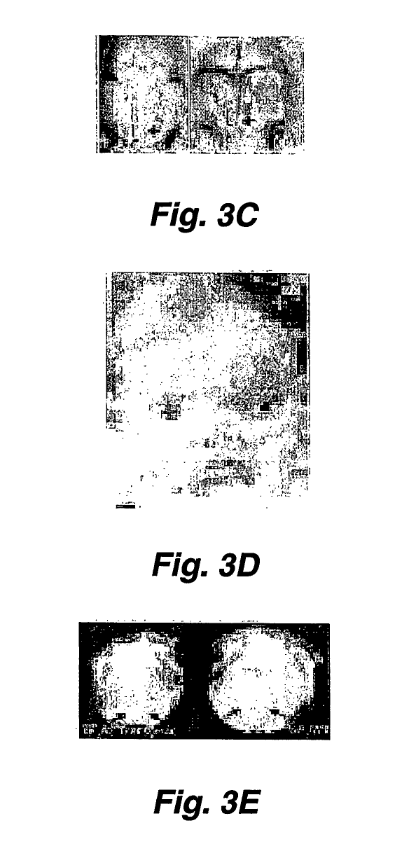

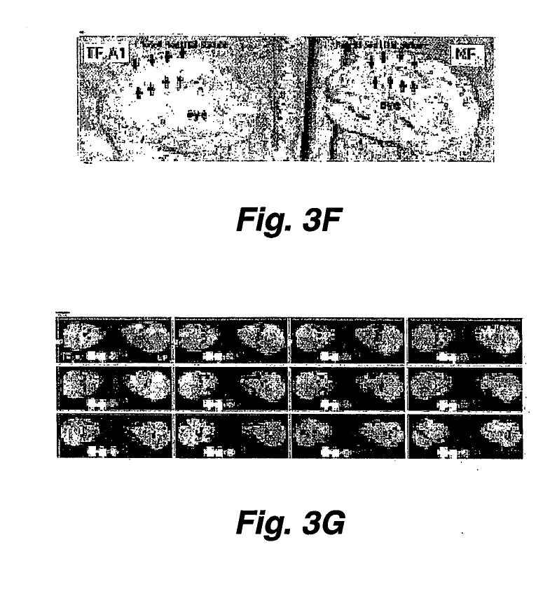

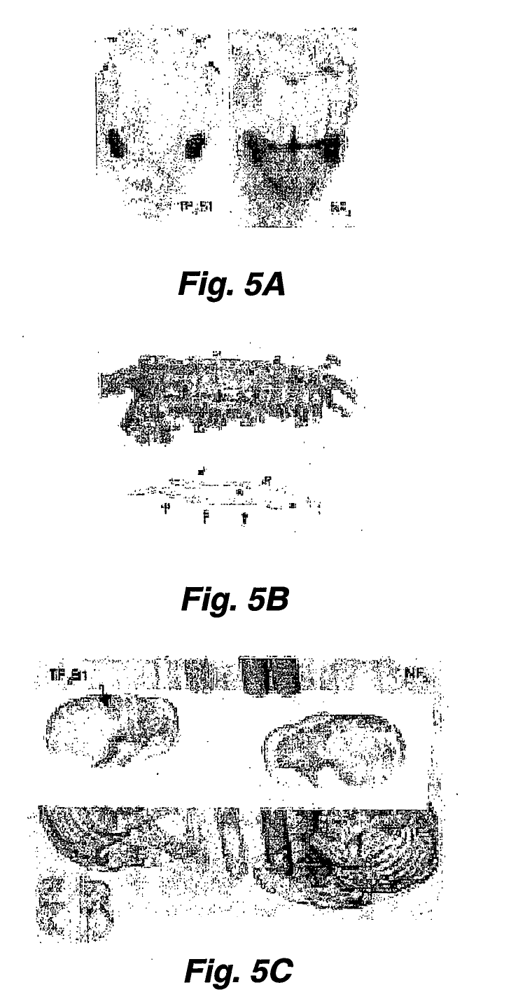

Three of the eight Nell-1 RNA-positive TF2 mice demonstrated severe craniofacial anomalies and died shortly after birth (see FIGS. 3A-3C, and FIG. 5). These mice also demonstrated detectable Nell-1 transgene expression in their total body mRNA (FIG. 2C) that was verified by Nell-1 immunostaining of skin, liver, and calvaria (FIG. 2D).

Morphological examination of one of the most severely affected TF2 mice revealed a large protuberance in the paramedial parietal area with completely closed sagittal and posterior-frontal (PF) sutures and partially closed coronal sutures (FIGS. 3A-3C). Clinically, this is similar to craniotelencephalic dysplasia, a form of human CS with premature sagittal, metopic, and coronal suture closure with secondary frontal bone bossing and paramedial encephalocele (FIG. 3D) (1). Brain MRI of this TF2 mouse revealed significantly reduced ventricle size and increased parenchymal edema, both of which are suggestive of increased intracranial pressures (FIG. 3E). Continued brain growth in the face of premature suture closure also generates increased intracranial pressures in humans with untreated CS. Microcomputerized tomography (MCT) scan and MRI analysis also demonstrated structural abnormalities in the cranium of this TF₂mouse (FIGS. 3F and 3G).

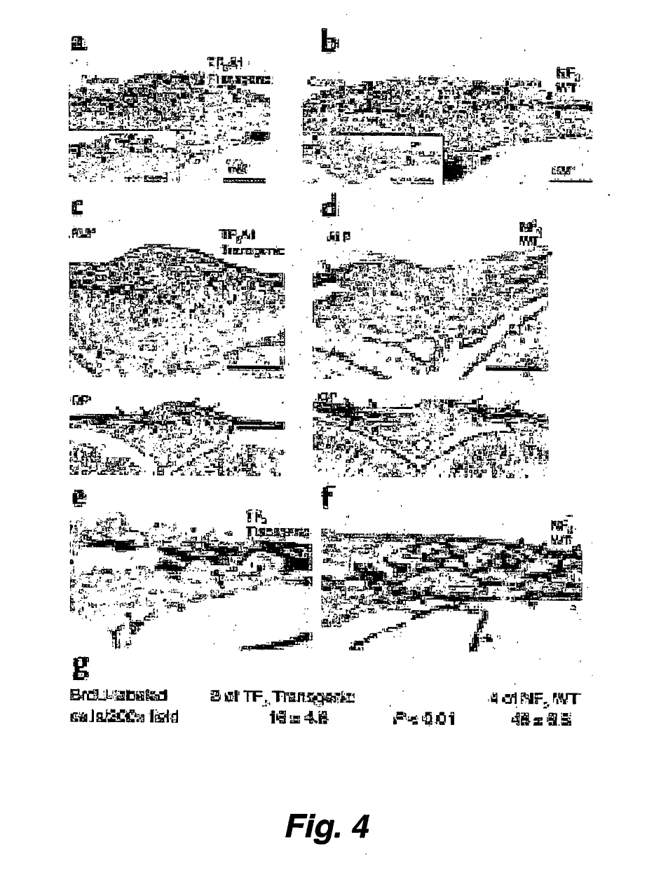

Histological examination of Nell-1 phenotype-positive TF2 mice revealed distinct differences from NF2 littermates. As in human CS, TF₂ mice displayed prematurely closing sutures seen histologically as thickened, disorganized ridges of calvarial ridges with closing/overlapping osteogenic fronts (FIG. 4 panels a and b). Whole-mount skeletal staining did not show any observable extracranial skeletal anomalies. Hematoxylin and eosin and tartrateresistant acid phosphatase staining of palatal and midmandible sutures, vertebrae, and long bones did not reveal any abnormal histology or increase in osteoclast number. Therefore, the effects of Nell-1 expression appear to be confined to the calvaria. Despite pan-tissue Nell-1 expression due to the use of the CMVpromoter, TF₂ mice exhibited cranial-specific anomalies that primarily affected calvarial suture patency and closure. Immunohistochemistry showed increased in vivo expression of osteoblastic differentiation markers (FIGS. 4 panels c and d).

In situ BrdU analysis of prematurely closing cranial sutures in Nell-1-expressing TF₂ mice demonstrated significantly reduced numbers of proliferating cells within osteogenic areas along suture edges (FIGS. 4 panels e and f). These data suggest that Nell-1 overexpression is associated with osteoblast differentiation. No statistically significant difference was observed in the total number (FIG. 4 Panel g) of cells per field along the sutures of TF₂ and NF2 mice. The observed decrease in proliferating cells may be secondary to the decreased proliferative abilities of differentiated osteoblasts or may reflect a primary defect in osteoblast proliferation.

Morphologic examination of a second severely affected TF2 animal showed significant cranial suture obliteration, primarily in the midline (i.e., sagittal and posterior frontal sutures), with bulging in the occipital (posterior) area. Overall, the skull was narrow and resembled those of humans with scaphocephaly and premature sagittal synostosis. MCT scanning revealed complete PF suture and partial sagittal and coronal suture closure (FIG. 5B). Histological correlation revealed marked calvarial bone overgrowth and overlap in the closed area of the sagittal suture (FIG. 5B).

To examine TF₂ embryologic development during gestation, two litters of ES15 TF₂ progeny were sacrificed. Nonviable littermates with exencephaly-like phenotypes were observed in two of 19 embryos. Interestingly, Liu et al. reported a similar finding of exencephaly for Msx2-overexpressing mice (6). The etiology for this phenotype is not clear. This result may also help to explain the observed low incidence of severely affected TF2 progeny among newborn mice.

Overexpression of Nell-1 in vitro Accelerates Osteoblast Differentiation.

Dysregulated bone formation has been proposed as a possible mechanism for calvarial overgrowth/ overlap and premature suture closure (18). Because abnormal suture site osteogenesis is the cardinal feature of Nell-1 TF₂ mice exhibiting premature suture closure, we hypothesized that Nell-1 overexpression may alter normal calvarial osteoblast cell cycling and differentiation pathways to promote premature osteogenesis.

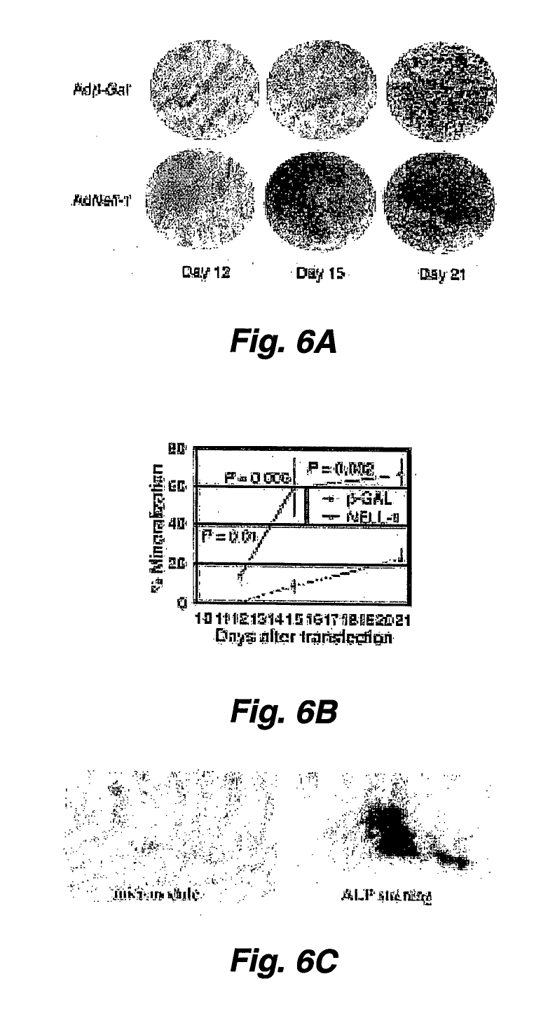

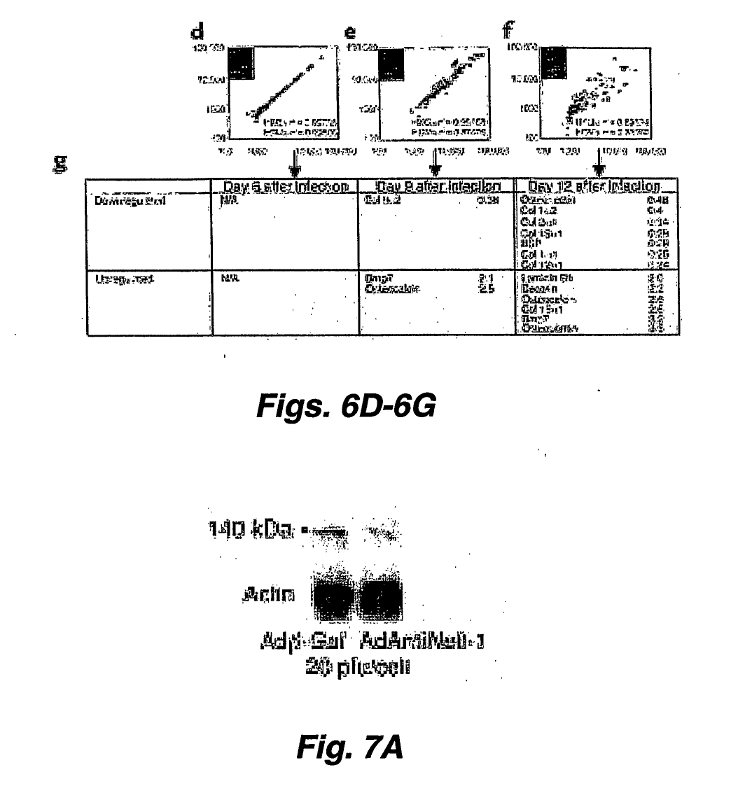

To test our hypothesis, we first examined the effect of Nell-1 on mineralization, a hallmark of osteoblast differentiation in vitro. Primary FRCC and MC3T3 (a mouse calvarial osteoblast-like cell line) cultures were infected with AdNell-1 at 20 pfu/cell in the presence of ascorbic acid. Ascorbic acid is essential for the induction and terminal differentiation/mineralization of osteoblasts (19). AdNell-1-infected FRCC and MC3T3 cultures mineralized more rapidly and profusely (more than sixfold) than Ad β-Gal-infected controls did (FIGS. 6A and 6B). In contrast, AdNell-1 infection did not elicit any mineralization response in NIH3T3, adult, or fetal rat primary fibroblast cells (data not shown). These data suggest that Nell-1 accelerates osteoblast mineralization and that the effects are osteoblast-specific.

Our previous in vivo BrdU results demonstrated significantly reduced cell proliferation along the osteogenic front in TF₂ mice. To determine whether Nell-1 overexpression in vitro also affects cell cycling, AdNell-1-infected MC3T3 cells (and Ad β-Gal controls, with and without ascorbic acid treatment and with and without 24 hours of serum starvation) were examined by flow cytometry at 24 and 48 hours after infection. No statistically significant changes were observed in populations in different phases of the cell cycle (two-tailed Student t test, P>0.05). The fact that MC3T3 cells did not demonstrate decreased proliferation after Nell-1 transfection may reflect inherent differences between in vivo and in vitro osteoblast cells or the influence of the extracellular milieu and stage of cellular differentiation.

Normal in vitro osteoblast differentiation is heralded by nodule formation (osteoblast cell aggregates) followed by mineralization. This differentiation program requires ascorbic acid. Interestingly, AdNell-1-infected MC3T3 cells, when cultured without ascorbic acid, also formed nodules expressing alkaline phosphatase beginning on day 3 after infection; control Ad β-Gal-infected cells did not. Nell-1-induced nodules in the absence of ascorbic acid, however, were smaller (≦20 cells per nodule, detectable at 100× magnification), and did not reveal mineralization with von Kossa staining (FIG. 6C). Moreover, late differentiation markers such as osteopontin were not expressed in these “micronodules.” The formation of micronodules by AdNell-1-infected osteoblasts in the absence of ascorbic acid suggests that Nell-1 alone may influence cell-cell adhesion but is not sufficient to induce full osteoblast differentiation.

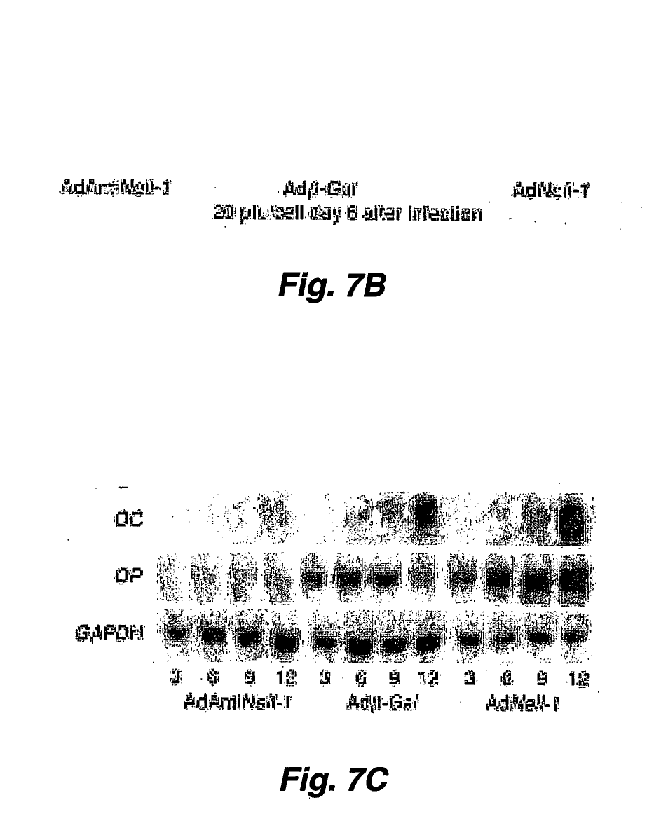

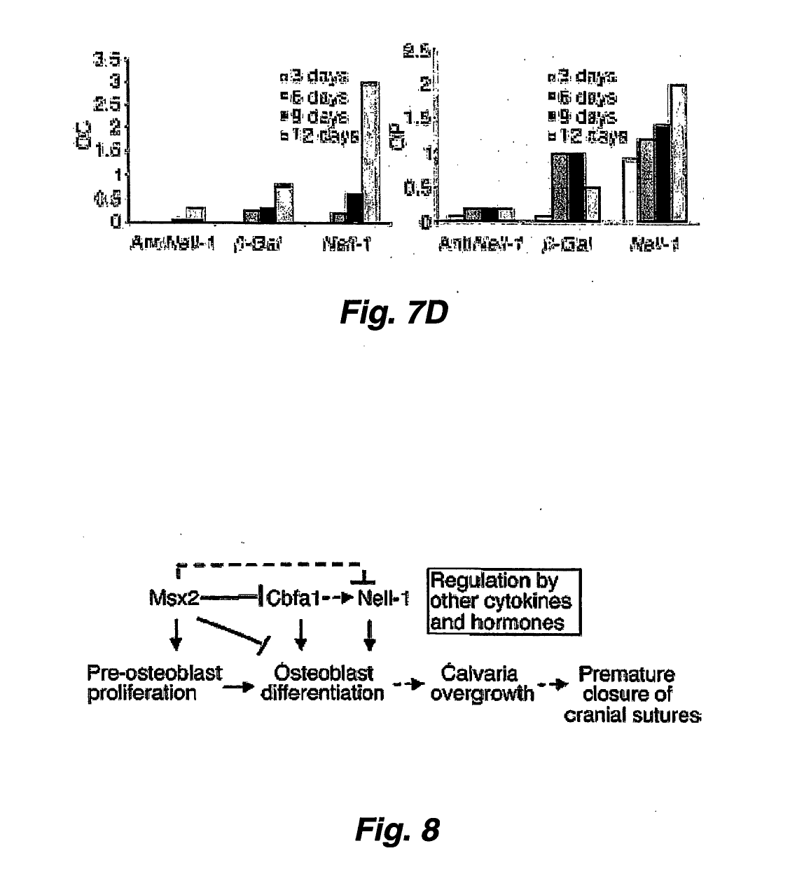

To prove that Nell-1 enhances osteoblast differentiation, RNA from AdNell-1-infected MC3T3 cells, cultured under normal conditions with ascorbic acid, were subjected to microarray analyses of various bone-specific markers at 6, 9, and 12 days after infection FIGS. 6D-6F). The purpose of the microarray was to determine whether AdNell-1-infected and control Ad β-Gal-infected cells demonstrated distinct differences in overall osteoblast differentiation marker expression patterns using regression analysis. By day 12, the expression pattern of osteoblast differentiation markers was distinctly different between AdNell-1-infected cells and Ad β-Gal-infected cells (r2=0.334). Microarray analyses used in this experiment were not meant to quantitate the expression of individual genes. Individual gene expression patterns should be interpreted with caution, e.g., genes with two or more fold up or downregulation should then be analyzed. Results should also be confirmed with RT-PCR or RNA analyses. Late differentiation markers, such as Bmp7, osteopontin, and osteocalcin, were upregulated more than twofold in AdNell-1-infected cells, while earlier markers, such as type I collagen and osteonectin, were downregulated more than twofold (FIG. 6G). This suggests that Nell-1 promotes osteoblast differentiation. Osteocalcin and osteopontin RNA upregulation were verified by RNA electrophoresis (see FIGS. 7C and 7D). Neither microarray nor reduced-cycle RT-PCR analyses demonstrated any significant changes in expression of Cbfa1, Tgf-β1,-β2, and -β3, or Tgf-βtypes-I, -II, and -III receptors, Fgfr1, or Fgfr2 in AdNell-1-infected MC3T3 cells (data not shown). This suggests that Nell-1 may operate downstream of these candidate genes or may affect distinctly different pathways.

Downregulation of Nell-1 in vitro Delays Osteoblast Differentiation.

To further address the physiologic function of Nell-1 in osteoblast differentiation, we tested the effect of downregulating the Nell-1 protein through adenoviral antisense Nell-1 infection in osteoblasts. FRCC cultures were infected with AdAntiNell-1 at 20 pfu/cell in the presence of ascorbic acid. AdAntiNell-1 downregulated Nell-1 protein expression to 40% of its normal expression level (FIG. 7A). FRCC cultures expressed significantly less alkaline phosphatase than did Ad β-Gal-infected controls or Ad Nell-1-infected cells (FIG. 7B). Osteocalcin and osteopontin RNA expression was also downregulated in AdAntiNell-1 cells (FIGS. 7C, and 7D). The ratio of osteocalcin in AdAntiNell-1-infected cells to osteocalcin in Ad β-Gal controls was less than 1:4 on day 9 and 1:2 on day 12 by Northern analysis. The ratio of osteopontin in AdAntiNell-1-infected cells to that in Ad β-Gal controls was less than 1:5 on days 6 and 9, and less than 2:5 on day 12. Therefore, knockdown data complement the overexpression data and suggest that Nell-1 plays an important role in osteoblast differentiation.

Discussion

NELL-1 is a relatively newly discovered molecule with previously unknown function. Because of the observed transient upregulation of NELL-1 during premature suture closure in CS patients (12), we simulated NELL-1 overexpression in a mouse model in order to investigate novel potential functions of Nell-1 in craniofacial development and pathology. We observed early suture closure and increased osteoblast differentiation in Nell-1 transgenic mice. Therefore, Nell-1 is likely a candidate for the control of local suture closure, and the overexpression of Nell-1 may play an important role in the mechanism of premature suture closure in CS. Based on our overexpression and knockdown in vitro data, Nell-1 most likely influences osteoblast differentiation. However, the molecular mechanism is unknown.

Nell-1 may induce osteoblast differentiation by binding and then sequestering or activating ligands, as well as by triggering receptor-mediated signaling (20). Nell-1's combination of chordin-like, cysteine-rich domains, NH₂-terminal thrombospondin-like module, and EGF-like repeats make it a likely modulator of growth factor activity. We have conducted preliminary studies to test this possibility. To determine whether Nell-1 binds to known EGF-like receptors, we previously added Nell-1 to IL-3-dependent cells expressing ErbB1, -2,-3, or -4. The addition of Nell-1 failed to produce tyrosine phosphorylation of these receptors. Nell-1, therefore, is not a ligand for these receptors even though Nell-1 is a known secretory protein with EGF-like repeats (13). Instead, Nell-1 may interact with other specific receptors that may be expressed only by certain cell types. Using the yeast two-hybrid system (14), we are in the process of isolating potential Nell-1 receptors. Because Nell-1 shares many motifs with thrombospondin-1 and chordin, it may hypothetically activate or sequester members of the TGF-β superfamily and function as a thrombospondin-1-like molecule to facilitate latent TGF-β1 activation (21). Recently, Abreu et al. suggested that Nell-1 is a member of the “chordin-like cysteine-rich domains” family, which includes chordin, kielin, crossveinless, twisted gastrulation (Tsg), and connective TGF (20). A common feature of the chordin-like cysteine-rich domains family members is that their expression is temporally and spatially specific, particularly in patterning. Another common feature is their interaction with members of the Bmp family and subsequent function as pro- or anti-Bmp's.

Specific Expression and Function of Nell-1 in vivo.

In our previous studies, we reported the earliest detectable Nell-1 expression in E11-E14 mice (12). Nell-1 is preferentially expressed in the craniofacial region, both prenatally and postnatally, during growth and development. Immunohistochemistry showed that Nell-1 localizes primarily to bone-forming areas of sutures and the calvarium and ossifying membranous bone in the mandible (data not shown). Both calvarial and mandibular membranous bones are thought to be neural crest derivatives (22). Preferential Nell-1 expression in the craniofacial region by neural crest derivatives suggests that Nell-1 may be important during skeletal craniofacial growth and development. Surprisingly, unlike other CS models involving generalized gene overexpression, Nell-1 transgenic mice displayed anomalies that were restricted to the calvarial bone, despite generalized, non-tissue-specific Nell-1 overexpression. This further supports our hypothesis that Nell-1 undergoes highly specific interaction to induce osteoblast differentiation. Nell-1 overexpression, therefore, is less likely to cause suture closure by nonspecifically perturbing the function of homologous molecules such as thrombospondin-1. This was verified by knockdown studies in vitro.

Effect of Nell-1 on Osteoblast Differentiation.

Normal osteoblasts cultured without ascorbic acid do not differentiate. Osteoblasts overexpressing Nell-1, on the other hand, form micronodules and express alkaline phosphatase in the absence of ascorbic acid. This suggests that Nell-1 alone is sufficient to induce some degree of osteoblast differentiation.

In addition, RNA microarray analyses of Nell-1 overexpression in osteoblasts cultured under normal conditions (i.e., with ascorbic acid) demonstrated upregulation of late differentiation markers at day 12 after transfection. AdNell-1-transfected osteoblasts also exhibited increased mineralization beginning on day 12 after transfection. These data indicate that Nell-1 may accelerate the rate of calvarial osteoblast differentiation and mineralization.

Nell-1 overexpression may not reflect the true physiological function of Nell-1, but rather the effect of Nell-1 overexpression on other thrombospondin-like molecules. Downregulation of Nell-1 clearly inhibited osteoblast differentiation. Nell-1 is therefore likely to be both sufficient and required for osteoblast differentiation in vitro. However, Nell-1 null mice need to be produced in order to justify this conclusion in vivo.

Nell-1's Relation to Currently Known CS Models.

Nell-1 overexpression produces craniofacial abnormalities similar to those resulting from Msx2 overexpression in vivo. Both mouse models exhibit suture overgrowth and an increased incidence of exencephaly. However, the cellular functions of these two genes appear to be distinctly different; continuous Msx2 overexpression induces proliferation and inhibits differentiation, while Nell-1 enhances differentiation. Mice with a Pro250 Arg mutation in Fgfr1, which induces Cbfa1 overexpression, have distinctly different phenotypes from mice overexpressing Nell-1 because calvarial fusion occurs much later (postnatal days 16-21) and gross suture overlap does not occur (8) in the mice with the Pro250 mutation. However, Cbfa1 has a similar cellular function to Nell-1 in vitro; both induce osteoblast differentiation with upregulation of bone marker genes. Nell-1 expression is modulated by Msx2 and Cbfa1. Cbfa1 transfection of FRCCs upregulated Nell-1 expression within 24 hours, while Msx2 transfection and Cbfa1/Msx2 cotransfection downregulated Nell-1 expression (unpublished observations). While all these candidate genes are important to the understanding of CS, Msx2 may be important in the earlier stages of CS (5, 6), while Fgfr1/Cbfa1 may play a role in the later stages of suture closure. Future investigation of the Nell-1 promoter, which contains conserved Cbfa1 and Msx binding sequences, may provide further understanding of their interactions (FIG. 8). These observations underscore the complexity of the dynamic genetic and environmental interactions in craniofacial growth and development.

In conclusion, we have created an animal model of human nonsyndromic CS by overexpressing Nell-1. Unlike other available CS models involving mutations in FGFRs or homeobox genes (1, 2, 8), our animal model exhibited anomalies that were localized to the craniofacial skeleton. We hypothesize that Nell-1 is sufficient, and probably required, to promote and accelerate calvarial osteoblast differentiation and bone formation. Mechanistically, Nell-1 overexpression induces intramembranous bone formation in cranial sutures and may lead to calvarial overgrowth/overlap and subsequent premature suture closure. Although Nell-1 has not yet been identified as a cause of CS in human genetic studies, the data strongly suggest that Nell-1 is part of a complex chain of events resulting in premature suture closure (1). The resemblance of Nell-1 transgenic mice to humans with nonsyndromic CS and Nell-l's association with known CS candidate genes provides new insights for CS research. Further investigation of the regulation and mechanism of Nell-1 in suture closure and bone formation can potentially accelerate our understanding of the cascade of events leading to premature suture closure in CS.

### REFERENCES

1. Cohen, M. M, Jr., and MacLean, R. E. 2000. Craniosynostosis: diagnosis, evaluation and management. 2nd edition. Oxford University Press. New York, N.Y., USA. 454 pp.

2. Coffin, J. D., et al. 1995. Abnormal bone growth and selective translational regulation in basic fibroblast growth factor (FGF-2) transgenic mice Mol. Biol. Cell. 6:1861-1873.

3. Carlton, M. B., Colledge, W. H., and Evans, M. J. 1998. Crouzon-like craniofacial dysmorphology in the mouse is caused by an insertional mutation at the Fgf3/Fgf4 locus. Dev. Dyn. 212:242-249.

4. Jabs, E. W., et al. 1993. A mutation in the homeodomain of the human MSX2 gene in a family affected with autosomal dominant craniosynostosis Cell. 75:443-450.

5. Liu, Y. H., et al. 1995. Premature suture closure and ectopic cranial bone in mice expressing Msx2 transgenes in the developing skull. Proc. Natl. Acad Sci. USA. 92:6137-6141.

6. Liu, Y. H., et al. 1999, Msx2 gene dosage influences the number of proliferative osteogenic cells in growth centers of the developing murine skull: a possible mechanism for MSX2-mediated craniosynostosis in humans Dev. Biol. 205:260-274.

7. Ma, L., Golden, S., Wu, L., and Maxson, R. 1996. The molecular basis of Boston-type craniosynostosis: the Pro148→His mutation in the N-terminal arm of the MSX2 homeodomain stabilizes DNA binding without altering nucleotide sequence preferences. Hum. Mol. Genet. 5:1915-1920.

8. Zhou, Y. X., et al. 2000. A Pro250Arg substitution in mouse Fgfr1 causes increased expression of Cbfa1 and premature fusion of calvarial sutures Hum. Mol. Genet. 9:2001-2008.

9. Otto, F., Kanegane, H., and Mundlos, S. 2002. Mutations in the RUNX2 gene in patients with cleidocranial dysplasia. Hum. Mutat. 19:209-216.

10. Matsuhashi, S., et al. 1996. New gene, nel, encoding a Mr 91 K protein with EGF-like repeats is strongly expressed in neural tissues of early stage chick embryos [erratum 1996, 207:233-234]. Dev. Dyn. 203:212-222.

11. Watanabe, T. K., et al. 1996. Cloning and characterization of two novel human cDNAs (NELL1 and NELL2) encoding proteins with six EGF-like repeats. Genomics. 38:273-276.

12. Ting, K., et al. 1999. Human NELL-1 expressed in unilateral coronal synostosis J. Bone Miner. Res. 14:80-89.

13.Kuroda, S., and Tanizawa, K. 1999. Involvement of epidermal growth factor-like domain of NELL proteins in the novel protein-protein interaction with protein kinase C. Biochem. Biophys. Res. Commun. 265:752-757.

14.Kuroda, S., et al. 1999. Biochemical characterization and expression analysis of neural thrombospondin-1-like proteins NELL1 and NELL2. Biochem. Biophys. Res. Commun. 265:79-86.

15. Hogan, B. C., Beddington, R., Constantini, F., and Lacy, E. 1986. Manipulating the mouse embryo: a laboratory manual. Cold Springs Harbor Laboratory Press. Cold Springs Harbor, N.Y., USA. 332 pp.

16. Laird, P. W., et al. 1991. Simplified mammalian DNA isolation procedure

Nucleic Acids Res. 19:4293.

17. Palmiter, R. D., Wilkie, T. M., Chen, H. Y., and Brinster, R. L. 1984. Transmission distortion and mosaicism in an unusual transgenic mouse pedigree Cell. 36:869-877.

18. Kim, H. J., Rice, D. P., Kettunen, P. J., and Thesleff, I. 1998. FGF-, BMP- and Shh-mediated signalling pathways in the regulation of cranial suture morphogenesis and calvarial bone development. Development. 125:1241-1251.

19. Franceschi, R. T., Iyer, B. S., and Cui, Y. 1994. Effects of ascorbic acid on collagen matrix formation and osteoblast differentiation in murine MC3T3-E1 cells. J. Bone Miner. Res. 9:843-854.

20. Garcia Abreu, J., Coffinier, C., Larrain, J., Oelgeschlager, M., and De Robertis, E. M. 2002. Chordin-like CR domains and the regulation of evolutionarily conserved extracellular signaling systems. Gene. 287:39-47.

21. Murphy-Ullrich, J. E., and Poczatek, M. 2000. Activation of latent TGF-beta by throinbospondin-1: mechanisms and physiology. Cytokine Growth Factor Rev. 11:59-69.

22. Hall, K. B., and Horstadius, S. 1988. The neural crest. =L Oxford University Press Oxford, United Kingdom. 305 pp.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of modulating calvarial osteoblast differentiation and mineralization in a human being, said method comprising:
altering expression or activity of Nell-1 in the human being, wherein increased expression or activity of Nell-1 increases osteoblast differentiation or mineralization and decreased expression or activity of Nell-1 decreases osteoblast differentiation or mineralization in the human being.

2. The method of claim 1, wherein Nell-1 expression or activity is inhibited by a method selected from the group consisting of an anti-Nell-1 antisense molecule, a Nell-1 specific riibozyme, a Nell-1 specific catalytic DNA, a Nell-1 specific RNAi, anti-Nell-1 intrabodies, and gene therapy approaches that knock out Nell-1 in target cells and/or tissues.

3. The method of claim 1, wherein Nell-1 expression or activity is increased by a method selected from the group consisting of transfecting a cell with an exogenous nucleic acid expressing Nell-1, and transfecting a cell with a Nell-1 protein.

4. The method of claim 2, wherein said Nell-1 expression or activity is inhibited in the human, and
wherein the human being is experiencing abnormal cranial suture development.

5. The method of claim 4, wherein said abnormal cranial suture development comprises craniosynostosis (CS).

6. A method of facilitating latent TGF-β1 activation in a human being, said method comprising administering exogenous Nell-1 to said human being, or increasing expression activity of endogenous Nell-1 in the human being.

7. A method of activating or sequestering a member of the TGF-β superfamily in a human being, said method comprising administering exogenous Nell-1 to said human being, or increasing expression activity of endogenous Nell-1 in the human being.

8. A method of screening for an agent that modulates osteoblast differentiation, said method comprising:
contacting a test cell which is a human cell containing a Nell-1 gene with a test agent; and
detecting a change in the expression level of a Nell-1 gene or the activity of Nell-1 in said test cell as compared to the expression of the Nell-1 gene or the activity of Nell-1 in a control cell where a difference in the expression level of Nell-1 or the activity of Nell-1 in the test cell and the control cell indicates that said agent modulates bone mineralization.

9. The method of claim 8, wherein said control is a negative control cell contacted with said test agent at a lower concentration than said test cell.

10. The method of claim 9, wherein said lower concentration is the absence of said test agent.

11. The method of claim 8, wherein said control is a positive control cell contacted with said test agent at a higher concentration than said test cell.

12. The method of claim 8, further comprising recording test agents that alter expression of the Nell-1 nucleic acid or the Nell-1 protein in a database of modulators of Nell-1 activity or in a database of modulators of bone mineralization.

13. The method of claim 8, wherein the expression level of Nell-1 is detected by measuring the level of Nell-1 mRNA in said cell.

14. The method of claim 13, wherein said level of Nell-1 mRNA is measured by hybridizing said mRNA to a probe that specifically hybridizes to a Nell-1 nucleic acid.

15. The method of claim 14, wherein said hybridizing is according to a method selected from the group consisting of a Northern blot, a Southern blot using DNA derived from the Nell-1 RNA, an array hybridization, an affinity chromatography, and an in situ hybridization.

16. The method of claim 15, wherein said probe is a member of a plurality of probes that forms an array of probes.

17. The method of claim 13, wherein said level of Nell-1 mRNA is measured using a nucleic acid amplification reaction.

18. The method of claim 8, wherein said level of Nell-1 is detected by determining the expression level of a Nell-1 protein in said biological sample.

19. The method of claim 18, wherein said detecting is via a method selected from the group consisting of capillary electrophoresis, a Western blot, mass spectroscopy, ELISA, immunochromatography, and immunohistochemistry.

20. The method of claim 8, wherein said cell is cultured ex vivo.

21. The method of claim 8, wherein said test agent is not an antibody.

22. The method of claim 8, wherein said test agent is not a protein.

23. A method of altering Nell-1 expression in a human cell, said method comprising altering the expression or activity of Msx2 and/or Cafa1 in the human cell.

24. The method of claim 23, comprising upregulating Cbfa1 expression or activity in the human cell to upregulate Nell-1 expression or activity.

25. The method of claim 23, comprising upregulating Msx2 expression or activity in the human cell to downregulate Nell-1 expression or activity.

26. A method of screening for an agent that modulates Nell-1 expression or activity in a human being, said method comprising:
contacting a test cell which is a human cell containing a Cbfa1 and/or an Msx2 gene with a test agent; and
detecting a change in the expression level of an Cbfa1 and/or an Msx2 gene or the activity of Cbfa1 and/or an Msx2 in said test cell as compared to the expression of the Cbfa1 and/or an Msx2 gene or the activity of Cbfa1 and/or an Msx2 in a control cell where a difference in the expression level of Cbfa1 and/or an Msx2 or the activity of Cbfa1 and/or an Msx2 in the test cell and the control cell indicates that said agent modulates Nell-1 expression or activity.

27. The method of claim 26, wherein said control is a negative control cell contacted with said test agent at a lower concentration than said test cell.

28. The method of claim 27, wherein said lower concentration is the absence of said test agent.

29. The method of claim 26, wherein said control is a positive control cell contacted with said test agent at a higher concentration than said test cell.

30. The method of claim 26, further comprising recording test agents that alter expression of Cbfa1 and/or an Msx2 gene or the activity of Cbfa1 and/or an Msx2 in a database of modulators of Nell-1 activity or in a database of modulators of bone mineralization.

31. The method of claim 26, wherein the expression level of Nell-1 is detected by measuring the level of Cbfa1 and/or an Msx2 mRNA in said cell.

32. The method of claim 31, wherein said level of Cbfa1 and/or an Msx2 mRNA is measured by hybridizing said mRNA to a probe that specifically hybridizes to a Cbfa1 and/or an Msx2 nucleic acid.

33. The method of claim 32, wherein said hybridizing is according to a method selected from the group consisting of a Northern blot, a Southern blot using DNA derived from the Cbfa1 and/or Msx2 RNA, an array hybridization, an affinity chromatography, and an in situ hybridization.

34. The method of claim 33, wherein said probe is a member of a plurality of probes that forms an array of probes.

35. The method of claim 31, wherein said level of Cbfa1 and/or Msx2 RNA is measured using a nucleic acid amplification reaction.

36. The method of claim 26, wherein said level of Cbfa1 and/or Msx2 is detected by determining the expression level of a Cbfa1 and/or Msx2 protein in said biological sample.

37. The method of claim 36, wherein said detecting is via a method selected from the group consisting of capillary electrophoresis, a Western blot, mass spectroscopy, ELISA, immunochromatography, and immunohistochemistry.

38. The method of claim 26, wherein said cell is cultured ex vivo.

39. The method of claim 26, wherein said test agent is not an antibody.

40. The method of claim 26, wherein said test agent is not a protein.

41. A pharmaceutical formulation comprising:
one or more active agents in an amount effective for increasing osteoblast differentiation or mineralization in a human being selected from the group consisting of a nucleic acid encoding a Nell-1 protein, a Nell-1 protein, and an agent that alters expression or activity of a Nell-1 protein; and
a pharmaceutically acceptable excipient.


SEQUENCE LISTING (2 sequences)

SEQ ID NO: 1 - DNA, 2977 bp, Homo sapiens
tagcaagtttggcggctccaagccaggcgcgcctcaggatccaggctcatttgcttccacctagcttcggtgccccctgctaggcggggaccctcgagagcgatgccgatggatttgattttagttgtgtggttctgtgtgtgcactgccaggacagtggtgggctttgggatggaccctgaccttcagatggatatcgtcaccgagcttgaccttgtgaacaccacccttggagttgctcaggtgtctggaatgcacaatgccagcaaagcatttttatttcaagacatagaaagagagatccatgcagctcctcatgtgagtgagaaattaattcagctgttccagaacaagagtgaattcaccattttggccactgtacagcagaagccatccacttcaggagtgatactgtccattcgagaactggagcacagctattttgaactggagagcagtggcctgagggatgagattcggtatcactacatacacaatgggaagccaaggacagaggcacttccttaccgcatggcagatggacaatggcacaaggttgcactgtcagttagcgcctctcatctcctgctccatgtcgactgtaacaggatttatgagcgtgtgatagaccctccagataccaaccttcccccaggaatcaatttatggcttggccagcgcaaccaaaagcatggcttattcaaagggatcatccaagatgggaagatcatctttatgccgaatggatatataacacagtgtccaaatctaaatcacacttgcccaacctgcagtgatttcttaagcctggtgcaaggaataatggatttacaagagcttttggccaagatgactgcaaaactaaattatgcagagacaagacttagtcaattggaaaactgtcattgtgagaagacttgtcaagtgagtggactgctctatcgagatcaagactcttgggtagatggtgaccattgcaggaactgcacttgcaaaagtggtgccgtggaatgccgaaggatgtcctgtccccctctcaattgctccccagactccctcccagtacacattgctggccagtgctgtaaggtctgccgaccaaaatgtatctatggaggaaaagttcttgcagaaggccagcggattttaaccaagagctgtcgggaatgccgaggtggagttttagtaaaaattacagaaatgtgtcctcctttgaactgctcagaaaaggatcacattcttcctgagaatcagtgctgccgtgtctgtagaggtcataacttttgtgcagaaggacctaaatgtggtgaaaactcagagtgcaaaaactggaatacaaaagctacttgtgagtgcaagagtggttacatctctgtccagggagactctgcctactgtgaagatattgatgagtgtgcagctaagatgcattactgtcatgccaatactgtgtgtgtcaaccttcctgggttatatcgctgtgactgtgtcccaggatacattcgtgtggatgacttctcttgtacagaacacgatgaatgtggcagcggccagcacaactgtgatgagaatgccatctgcaccaacactgtccagggacacagctgcacctgcaaaccgggctacgtggggaacgggaccatctgcagagctttctgtgaagagggctgcagatacggtggaacgtgtgtggctcccaacaaatgtgtctgtccatctggattcacaggaagccactgcgagaaagatattgatgaatgttcagagggaatcattgagtgccacaaccattcccgctgcgttaacctgccagggtggtaccactgtgagtgcagaagcggtttccatgacgatgggacctattcactgtccggggagtcctgtattgacattgatgaatgtgccttaagaactcacacctgttggaacgattctgcctgcatcaacctggcagggggttttgactgtctctgcccctctgggccctcctgctctggtgactgtcctcatgaaggggggctgaagcacaatggccaggtgtggaccttgaaagaagacaggtgttctgtctgctcctgcaaggatggcaagatattctgccgacggacagcttgtgattgccagaatccaagtgctgacctattctgttgcccagaatgtgacaccagagtcacaagtcaatgtttagaccaaaatggtcacaagctgtatcgaagtggagacaattggacccatagctgtcagcagtgtcggtgtctggaaggagaggtagattgctggccactcacttgccccaacttgagctgtgagtatacagctatcttagaaggggaatgttgtccccgctgtgtcagtgacccctgcctagctgataacatcacctatgacatcagaaaaacttgcctggacagctatggtgtttcacggcttagtggctcagtgtggacgatggctggatctccctgcacaacctgtaaatgcaagaatggaagagtctgttgttctgtggattttgagtgtcttcaaaataattgaagtatttacagtggactcaacgcagaagaatggacgaaatgaccatccaacgtgattaaggataggaatcggtagtttggtttttttgtttgttttgtttttttaaccacagataattgccaaagtttccacctgaggacggtgtttcggaggttgccttttggacctaccactttgctcattcttgctaacctagtctaggtgacctacagtgccgtgcatttaagtcaatggttgttaaaagaagtttcccgtgttgtaaatcatgtttcccttatcagatcatttgcaaatacatttaaatgatctcatggtaaatggttgatgtattttttgggtttattttgtgtactaaccataatagagagagactcagctccttttatttattttgttgatttatggatcaaattctaaaataaagttgcctgttgtgactttt

SEQ ID NO: 2 - DNA, 1722 bp, Homo sapiens
  misc_feature: n is a, c, g, or t
gatcagtgctgccgtgtctgtagaggtcataacttttgtgcagaaggacctaaatgtggtgaaaactcagagtgcaaaaactggaatacaaaagctacttgtgagtgcaagagtggttacatctctgtccaggggagactctgcctactgtgaagatattgatgagtgtgcagctaagatgcattactgtcatgccaatactgtgtgtgtcaaccttcctgggttatatcgctgtgactgtgtcccaggatacattcgtgtggatgacttctcttgtacagaacacgatgaatgtggcagcggccagcacaactgtgatgagaatgccatctgcaccaacactgtccagggacacagctgcacctgcaaaccgggctacgtggggaacgggaccatctgcagagctttctgtgaagagggctgcagatacggtggaacgtgtgtggctcccaacaaatgtgtctgtccatctggattcacaggaagccactgcgagaaagatattgatgaatgttcagagggaatcattgagtgccacaaccattcccgctgcgttaacctgccagggtggcaccactgtgagtgcagaagcggtttccatgacgatgggacctattcactgtccggggagtcctgtattgacattgatgaatgtgccttaagaactcacacctgttggaacgattctgcctgcatcaacctggcagggggttttgactgtctctgcccctgtgggccctcctgctctggtgactgtcctcatgaaggggggctgaagcacaatggccaggtgtggaccttgaaagaagacaggtgttctgtctgctcctgcaaggatggtaagatattctgccgacggacagcttgtgattgccagaatccaagtgctgacctattctgttgcccagaatgtgacaccagagtcacaagtcaatgtttagaccaaaatggtcacaagctgtatcgaagtggagacaattggacccatagctgtcagcagtgtcggtgtctggaaggagaggtagattgctggccactcacttgccccaacttgagctgtgagtatacagctatcttagaaggggaatgttgtccccgctgtgtcagtgacccctgcctagctgataacatcacctatgacatcagaaaaacttgcctggacagtatggtgtttcacggcttagtggctcagtgtggacgatggctggatctccctgcacaacctgtaaatgcaagaatggaagagtctgttgttctgtggattttgagtgtcttcaaaataattgaagtatttacagtggactcaacgcagaagaatggacgaaatgaccatccaacgtgattaaggataggaatcggtagtttggtttttttgtttgttttgtttttttaaccacagataattgccaaagtttccacctgaggacggtgtttggaggttgccttttggacctaccactttgctcattcttgctaacctagtttaggtgacctacagtgccgtgcatttaagtcagtggttgttaaaagaagtttcccgcgttgtaaatcatgtttcccttatcagatcatttgcaaatacatttaaatgatntcatggtaaatgttgctgtattttttggtttattttctgtactaacataatagagagagantnagctccttttatttattttgttgatttatggatcaaattntaaaataaagttgcctgttgtgnaa